(19) United States
(12) Patent Application Publication
(10) Pub. No.: US 2013/0149237 A1
(43) Pub. Date: Jun. 13, 2013

(54) DELIVERY SYSTEM FOR DIAGNOSTIC AND THERAPEUTIC AGENTS

(71) Applicants: Mark Dennis, San Carlos, CA (US); Per-Ola Freskgard, Reinach BL (CH); Stephen Tam, Belmont, CA (US); Ryan Watts, San Mateo, CA (US); Joy Yu, San Francisco, CA (US)

(72) Inventors: Mark Dennis, San Carlos, CA (US); Per-Ola Freskgard, Reinach BL (CH); Stephen Tam, Belmont, CA (US); Ryan Watts, San Mateo, CA (US); Joy Yu, San Francisco, CA (US)

(73) Assignees: GENENTECH, INC., South San Francisco, CA (US); F. HOFFMANN-LA ROCHE AG, Basel (CH)

(21) Appl. No.: 13/574,584

(22) PCT Filed: Jan. 21, 2011
(86) PCT No.: PCT/US2011/022124

§ 371 Date: Feb. 15, 2013

Related U.S. Application Data
(60) Provisional application No. 61/297,676, filed on Jan. 22, 2010.

(51) Int. Cl. A61K 47/48; A61K 39/395

(52) U.S. Cl. CPC A61K 47/48369; A61K 39/3955
USPC 424/1.49; 424/172.1; 530/391.7; 424/136.1; 424/143.1; 424/133.1; 424/178.1

(57) ABSTRACT

The invention provides shuttle agents and methods of using the same to facilitate the translocation of therapeutic or diagnostic molecules into the CNS.

### FIELD OF THE INVENTION

The present invention relates to LRP8-binding shuttle agents and methods of using the same. The invention also relates to methods of facilitating the translocation of therapeutic or diagnostic molecules into the CNS.

### BACKGROUND

Neurological disorders represent a major cause of mortality and disability globally. Despite extensive progress, current treatment options remain limited in many aspects. One reason for this limitation is that the brain allows access only to certain types of molecules. While this limited access facilitates protection of the brain, it also means that many potentially useful compounds are unable to penetrate into the central nervous system (CNS) and are therefore unable to exert therapeutic activity in or be used in the diagnosis of certain neurological disorders or other diseases of the CNS. The present invention represents an advance in providing targeted accessibility of the CNS for desired molecules whose ability to cross the blood-brain barrier (BBB) is otherwise limited. Further, the present invention describes approaches that achieve high brain selectivity in delivery of such molecules.

### SUMMARY

The invention provides compositions and methods to facilitate the delivery of therapeutic and diagnostic compounds into the CNS across the blood-brain barrier. In one embodiment, the invention provides a composition comprising an LRP8-binding molecule and at least one CNS-active compound. In one aspect, the LRP8-binding molecule is conjugated to the at least one CNS-active compound. In one such aspect, the conjugation is a covalent linkage between the LRP8-binding molecule and the at least one CNS-active compound. In another such aspect, the conjugation is by a linker. In another aspect, the LRP8-binding molecule is selected from a natural ligand of LRP8, a fragment of a natural ligand of LRP8, a modified form of a natural ligand of LRP8, and a fragment of a modified form of a natural ligand of LRP8. In one such aspect, the natural ligand of LRP8 is selected from reelin and selenoprotein P. In another aspect, the LRP8-binding molecule is an antibody. In one such aspect, the antibody is a multispecific antibody. In an [symbol] from a monoclonal antibody, a human antibody, a humanized antibody, a chimeric antibody, and an antibody fragment that binds LRP8.

In a further aspect, the LRP8-binding molecule does not compete with one or more natural ligands of LRP8 for binding to LRP8. In a further aspect, the LRP8-binding molecule competes with one or more natural ligands of LRP8 for binding to LRP8. In a further aspect, the LRP8-binding molecule binds to the extracellular domain of LRP8. In a further aspect, the LRP8-binding molecule preferentially binds to LRP8 expressed in the brain.

In a further aspect, the CNS-active compound is selected from a therapeutic compound and a diagnostic compound. In one such aspect, the therapeutic compound is selected from a neurotrophic factor and a compound to treat or prevent one or more of neuropathy, amyloidosis, cancer, an ocular disease or disorder, viral or microbial infection, inflammation, ischemia, neurodegenerative disease, seizure, a behavioral disorder, and a lysosomal storage disease. In one such aspect, the therapeutic compound is selected from a compound to treat Parkinson's disease and a compound to treat Alzheimer's disease. In another such aspect, the diagnostic compound is a labeled peptide or antibody that specifically binds to a CNS target. In a further aspect, binding of the LRP-binding molecule to LRP8 effects the transport of the CNS-active compound across the blood-brain barrier.

In a further embodiment, the invention provides a pharmaceutical formulation comprising a composition comprising an LRP8-binding molecule and at least one CNS-active compound and a pharmaceutically acceptable carrier. In one aspect, the pharmaceutical formulation further comprises an additional therapeutic agent.

In a further embodiment, the invention provides a method for modulating the transport of a CNS-active compound across the blood-brain barrier in a mammal by modulating the expression, stability, or activity of LRP8. In one aspect, the targeting is by means of an LRP8-binding molecule and transport of the CNS-active compound is increased. In another aspect, the LRP8-binding molecule and the CNS-active compound are administered to the mammal simultaneously.

In another aspect, the LRP8-binding molecule is conjugated to the CNS-active compound. In one such aspect, the conjugation between the LRP8-binding molecule and the CNS-active compound is selected from covalent association, association with a linker, and as different binding moieties within the same multispecific antibody.

In a further aspect, the LRP8-binding molecule is selected from a natural ligand of LRP8, a fragment of a natural ligand of LRP8, a modified form of a natural ligand of LRP8, and a fragment of a modified form of a natural ligand [symbol] ligand of LRP8 is selected from reelin and selenoprotein P. In a further aspect, the LRP8-binding molecule is an antibody. In one such aspect, the antibody is a multispecific antibody. In another such aspect, the antibody is selected from a monoclonal antibody, a human antibody, a humanized antibody, a chimeric antibody, and an antibody fragment that binds LRP8.

In a further aspect, the LRP8-binding molecule does not compete with one or more natural ligands of LRP8 for binding to LRP8. In a further aspect, the LRP8-binding molecule competes with one or more natural ligands of LRP8 for binding to LRP8. In a further aspect, the LRP8-binding molecule binds to the extracellular domain of LRP8. In a further aspect, the LRP8-binding molecule preferentially binds to LRP8 expressed in the brain.

In a further aspect, the CNS-active compound is selected from a therapeutic compound and a diagnostic compound. In one such aspect, the therapeutic compound is selected from a neurotrophic factor and a compound to treat or prevent one or more of neuropathy, amyloidosis, cancer, an ocular disease or disorder, viral or microbial infection, inflammation, ischemia, neurodegenerative disease, seizure, a behavioral disorder, and a lysosomal storage disease. In another such aspect, the therapeutic compound is selected from a compound to treat Parkinson's disease and a compound to treat Alzheimer's disease. In another such aspect, the diagnostic compound is a labeled peptide or antibody that specifically binds to a CNS target. In a further aspect, the mammal is a human.

In a further aspect, the LRP8-binding molecule and the CNS-active compound are administered in conjunction with one or more additional therapeutic agents. In one such aspect, the LRP8-binding molecule and the CNS-active compound are administered in conjunction with a pharmaceutically-acceptable carrier.

In another embodiment, the invention provides a method for modulating the transport of a CNS-active compound across a vascular endothelial cell layer including tight junctions by targeting LRP8. In another embodiment, the invention provides a method of treating an individual having a CNS disease or CNS disorder comprising administering to the individual an effective amount of the composition or pharmaceutical formulation of any of the foregoing compositions or pharmaceutical formulations.

In another embodiment, the invention provides a method of decreasing or preventing the severity, duration, or symptoms of a CNS disease or CNS disorder in an individual suffering therefrom comprising administering to the individual an effective amount of the composition or pharmaceutical formulation of any of the foregoing compositions or pharmaceutical formulations.

In another embodiment, the invention provides [symbol] or CNS disorder in an individual comprising administering to the individual an effective amount of the composition or pharmaceutical formulation of any of the foregoing compositions or pharmaceutical formulations, visualizing or quantifying the CNS-active compound in the brain of the individual, and comparing the results to control results from individuals with known instance of the CNS disease or disorder or lack thereof.

In another embodiment, the invention provides a method of staging a CNS disease or CNS disorder in an individual comprising administering to the individual an effective amount of the composition or pharmaceutical formulation of any of the foregoing compositions or pharmaceutical formulations, visualizing or quantifying the CNS-active compound in the brain of the individual, and comparing the results to control results from individuals with known stages of the CNS disease or CNS disorder.

In another embodiment, the invention provides an LRP8-binding molecule or a composition comprising an LRP8-binding molecule and at least one CNS-active compound for use as a medicament. In one aspect, the LRP8-binding molecule or the composition comprising an LRP8-binding molecule is one of the foregoing LRP8-binding molecules or compositions comprising an LRP8-binding molecule. In one aspect, the LRP8-binding molecule is conjugated to the at least one CNS-active compound. In one such aspect, the conjugation is a covalent linkage between the LRP8-binding molecule and the at least one CNS-active compound. In another such aspect, the conjugation is by a linker.

In a further aspect, the LRP8-binding molecule is selected from a natural ligand of LRP8, a fragment of a natural ligand of LRP8, a modified form of a natural ligand of LRP8, and a fragment of a modified form of a natural ligand of LRP8. In one such aspect, the natural ligand of LRP8 is selected from reelin and selenoprotein P. In a further aspect, the LRP8-binding molecule is an antibody. In one such aspect, the antibody is a multispecific antibody. In another such aspect, the antibody is selected from a monoclonal antibody, a human antibody, a humanized antibody, a chimeric antibody, and an antibody fragment that binds LRP8.

In a further aspect, the LRP8-binding molecule does not compete with one or more natural ligands of LRP8 for binding to LRP8. In a further aspect, the LRP8-binding molecule competes with one or more natural ligands of LRP8 for binding to LRP8. In a further aspect, the LRP8-binding molecule binds to the extracellular domain of LRP8. In a further aspect, the LRP8-binding molecule preferentially binds to LRP8 expressed in the brain.

In a further aspect, the CNS-active compound is selected from a therapeutic compound and a diagnostic compound. In one such aspect, the therapeutic compound is selected from a neurotrophic factor and a compound to treat or preven [symbol] amyloidosis, cancer, an ocular disease or disorder, viral or microbial infection, inflammation, ischemia, neurodegenerative disease, seizure, a behavioral disorder, and a lysosomal storage disease. In another such aspect, the therapeutic compound is selected from a compound to treat or prevent Parkinson's disease and a compound to treat or prevent Alzheimer's disease. In another such aspect, the diagnostic compound is a labeled peptide or antibody that specifically binds to a CNS target. In a further aspect, binding of the LRP-binding molecule to LRP8 effects the transport of the CNS-active compound across the blood-brain barrier.

In another embodiment, the invention provides a pharmaceutical formulation comprising a composition comprising an LRP8-binding molecule and at least one CNS-active compound for use as a medicament and a pharmaceutically acceptable carrier. In one aspect, the pharmaceutical formulation further comprises an additional therapeutic agent.

In another embodiment, the invention provides the use of an LRP8-binding molecule or a composition comprising an LRP8-binding molecule for the manufacture of a medicament for treatment of a CNS disease or CNS disorder. In one aspect, the LRP8-binding molecule or the composition comprising an LRP8-binding molecule is one of the foregoing LRP8-binding molecules or compositions comprising an LRP8-binding molecule. In another embodiment, the invention provides the use of an LRP8-binding molecule or a composition comprising an LRP8-binding molecule for the manufacture of a medicament for diagnosing or staging a CNS disease or CNS disorder. In one aspect, the LRP8-binding molecule or the composition comprising an LRP8-binding molecule is one of the foregoing LRP8-binding molecules or compositions comprising an LRP8-binding molecule.

### BRIEF DESCRIPTION OF THE FIGURES

FIG. 1 is a schematic diagram of the structure of the LRP8 protein in the context of a cell membrane. The extracellular domain of LRP8 is the portion of LRP8 extending from the N-terminus of the protein to the transmembrane domain, and encompasses 7 ligand-binding repeats and an EGF-precursor domain. The intracellular portion of LRP8 encompasses the intracellular signaling domain and is present in the interior of the cell.

FIGS. 2A-2D depict the results of experiments identifying LRP8 as a protein highly and specifically expressed on brain vascular endothelial cells associated with the blood-brain barrier. FIG. 2A depicts the results of FACS analyses to sort CD31-positive endothelial cells from other types of cells for further experimentation. FIGS. 2B and 2C graphically depict results of the microarray experiments described in Example 1. In FIGS. 2B-1-2B-3, the relative transcript levels from the adult samples are sh [symbol] from the same analysis and include adult, pup and embryo samples for LRP8 and LRP1 only. The maximum Y-axis value on the LRP8 graph is 5.7 while the maximum Y-axis value on the LRP1 graph is 0.123. FIG. 2D shows the results of the confirmatory quantitative RT-PCR experiments described in Example 1, demonstrating that virtually no LRP8 RNA is observed in lung/liver vascular endothelial cells, while those cells do show high levels of LRP1 RNA. Relative transcript adundance values are plotted with all values normalized to a mean brain level set at 1.

FIGS. 3A-3E depict the results of the experiments described in Example 2A to identify reagents useful for and to determine the detectability of LRP8 protein in vascular endothelial cell samples. FIG. 3A shows Western blot analyses of ApoER2 using different anti-ApoER2 antibodies. Recombinant human ApoER2 was diluted into Invitrogen LDS sample buffer with 1× reducing agent and loaded onto a 10% Bis-Tris Nupage 15-well gel in 10 ng, 3 ng, ing, 0.3 ng amounts. Left panel: a.) anti-ApoER2 mouse monoclonal antibody (Abcam ab58216) diluted 1:500 (2.0 μg/ml) was used as the primary antibody and incubated with the blot for 48 hours at 4° C. on a rotator, b.) biotinXX-anti-mouse antibody diluted 1:2000 was used as the secondary antibody and incubated with the blot for 2 hours at room temperature. Center panel: a.) rabbit anti-ApoER2 rabbit ployclonal antibody (Invitrogen 40-7800) was used as the primary antibody and incubated with the blot for 48 hours at 4° C. on a rotator, b.) biotinXX-anti-rabbit antibody diluted 1:2000 was used as the secondary antibody and incubated with the blot for 2 hours at room temperature. Right panel: a.) LRP8 mouse polyclonal antibody (Abnova H00007804-A01) diluted 1:500 (2.0 μg/ml) was used as the primary antibody and incubated with the blot for 48 hours at 4° C. on a rotator, 2.) biotinXX-anti-mouse antibody diluted 1:2000 was used as the secondary antibody and incubated with the blot for 2 hours at room temperature. FIGS. 3B and 3C show Western blots as in FIG. 3A, but assessing LRP8 presence in D3 cell lysates from passage 30 of T175 flask cultures. FIG. 3D depicts the results of Western analyses detecting the presence of LRP8 protein in HUVEC or HBMEC cell lysates, as described in Example 2. The left panel used rabbit anti-human LRP8 antibody (Zymed), and the right panel used goat anti-human LRP8 antibody (Novus), and both detected the presence of bands in the 98 KD molecular weight range, expected to correlate with the presence of LRP8. FIG. 3E graphically shows the results of a competition assay demonstrating that RAP does not compete with the anti-LRP8 antibody for binding to ApoER2.

FIGS. 4A-4F depict the results of experiment [symbol] localization of LRP8 in various cultured vascular endothelial cells and in mouse and human tissues. FIG. 4A shows images from immunocytochemical analyses of anti-LRP8 binding (left panels, Santa Cruz goat anti-human LRP8 antibody; right panels Zymed rabbit anti-human LRP8 antibody) to fixed, permeabilized HUVEC. FIG. 4B shows immunofluorescence images obtained by staining immortalized human brain endothelial cells (hCMEC/D3, left) or primary human brain microvascular endothelial cells (right) with anti-human LRP8 antibody (Sigma A3481) after fixation/permeabilization (secondary antibody goat-anti-rabbit Alexa-Fluor488). Both images show a membrane and vesicular distribution of LRP8; the expression level is higher in primary cells than in the immortalized cell line. FIG. 4C shows images from immunocytochemical analyses of the effects of pretreatment of HUVEC with the LRP8 ligand reelin on anti-LRP8 antibody staining of those cells, and demonstrates that reelin treatment does not change the localization of LRP8 on HUVEC. FIG. 4D provides images from the immunohistochemical analyses of mouse brain tissue labeled with anti-LRP8 and CD31, showing that LRP8 colocalizes with vascular endothelial cells. FIG. 4E provides images showing anti-LRP8 antibody localization in brain sections from mice systemically treated with Alexa488-labeled anti-LRP8 antibody for one hour. The antibody can clearly be seen throughout the section in a punctate pattern and also is readily visualized in the blood vessels. FIG. 4F shows fluorescence micrographs of human cortical Alzheimer's disease brain sections with primary antibody against LRP8 together with DAPI staining for cell nuclei. LRP8 staining at the site of blood vessels can be detected.

FIG. 5A schematically depicts the transmigration assay described in Example 3. FIGS. 5B-5C depict the results of experiments described in Example 3 to determine the ability of LRP8 to translocate across hCMEC/D3 cell monolayers in an in vitro transmigration assay.

### DETAILED DESCRIPTION OF EMBODIMENTS OF THE INVENTION

### I. Definitions

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid change [symbol] less, 6 or less, 5 or less, 4 or less, 3 or less, or 2 or less. In some embodiments, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The terms “anti-LRP8 antibody” and “an antibody that binds to LRP8” refer to an antibody that is capable of binding LRP8 with sufficient affinity such that the antibody is useful as an agent in targeting LRP8 and facilitating the LRP8-mediated translocation of associated or proximal molecules. In one embodiment, the extent of binding of an anti-LRP8 antibody to an unrelated, non-LRP8 protein is less than about 10% of the binding of the antibody to LRP8 as measured, e.g., by a radioimmunoassay (RIA). In certain embodiments, an antibody that binds to LRP8 has a dissociation constant (Kd) of ≦104, ≦100 nM, ≦10 nM, ≦1 nM, ≦0.1 nM, ≦0.01 nM, or ≦0.001 nM (e.g. 10⁻⁸ M or less, e.g. from 10⁻⁸ M to 10⁻¹³ M, e.g., from 10⁻⁹ M to 10⁻¹³ M). In certain embodiments, an anti-LRP8 antibody binds to an epitope of LRP8 that is conserved among LRP8 from different species. In certain embodiments, an anti-LRP8 antibody binds to an extracellular epitope of LRP8. In certain embodiments, an anti-LRP8 antibody does not compete for binding to LRP8 with one or more natural LRP8 ligands. In certain embodiments, an anti-LRP8 antibody does compete for binding to LRP8 with one or more natural LRP8 ligands.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule ot [symbol] comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The “blood-brain barrier” or “BBB” refers to the physiological barrier between the peripheral circulation and the brain and spinal cord which is formed by tight junctions within the brain capillary endothelial plasma membranes, creating a tight barrier that restricts the transport of molecules into the brain, even very small molecules such as urea (60 Daltons). The blood-brain barrier within the brain, the blood-spinal cord barrier within the spinal cord, and the blood-retinal barrier within the retina are contiguous capillary barriers within the CNS, and are herein collectively referred to a the blood-brain barrier or BBB. The BBB also encompasses the blood-CSF barrier (choroid plexus) where the barrier is comprised of ependymal cells rather than capillary endothelial cells.

The “central nervous system” or “CNS” refers to the complex of nerve tissues that control bodily function, and includes the brain and spinal cord.

The term “chimeric” protein refers to a molecule where one portion of the protein derives from a particular source or species, while the remainder of the protein derives from a different source or species. In the context of an antibody, a “chimeric antibody” refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgAᵢ, and IgA₂. The heavy chain constant domains that correspond to the different classes of immunoglobulins are called α, δ, ε, γ, and μ, respectively.

A “central nervous system active compound” or “CNS-active compound” as used herein refers to a substance that has an effect within the CNS of a subject. CNS active compounds include, but are not limited to, therapeutic [symbol] compounds with an effect useful in research. Therapeutic CNS-active compounds are compounds that are effective to treat one or more CNS diseases or disorders, to prevent the onset or development of one or more CNS diseases or disorders, or to decrease or prevent the severity, duration, or symptoms of one or more CNS diseases or disorders. Diagnostic CNS active compounds are compounds that are effective in diagnosing or staging one or more CNS diseases or disorders, or in imaging one or more areas of the brain.

A “central nervous system disease or disorder” or “CNS disease or disorder” as used herein refers to a disease or disorder which affects the CNS and/or which has an etiology in the CNS. Exemplary CNS diseases or disorders include, but are not limited to, neuropathy, amyloidosis, cancer, an ocular disease or disorder, viral or microbial infection, inflammation, ischemia, neurodegenerative disease, seizure, behavioral disorders, and a lysosomal storage disease. For the purposes of this application, the CNS will be understood to include the eye, which is normally sequestered from the rest of the body by the blood-retina barrier.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

An “effective amount” of an agent, e.g., a pharmaceutical formulation, refers to an amount effective, at dosages and for periods of time necessary, to achieve the desired therapeutic or prophylactic result.

The term “Fc region” herein is used to define a [symbol] immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. [symbol] subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, Md. (1991).) With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008).) Unless otherwise indicated, HVR residues and other residues in the variable domain (e.g., FR residues) are numbered herein according to Kabat et al., supra.

An “immunoconjugate” is an antibody conjugated to one or more heterologous molecule(s).

An “individual” or “subject” is a mammal. M [symbol] domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated” polypeptide is one which has been separated from a component of its natural environment. In some embodiments, a polypeptide is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of, e.g., antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-LRP8 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

A “linker moiety” or “linker,” as used herein, refers to a structure that covalently or non-covalently connects the LRP8-binding compound to a CNS-active molecule. The main function of a linker is as a spacer, and its presence in the shuttle agents of the invention is optional, depending on the needs of the particular LRP8-binding molecule and CNS-active compound to be conjugated. In certain embodiments, a linker is a peptide. In other embodiments, a linker is a chemical linker.

The term “LRP8,” used interchangeably with “apolipoprotein E receptor 2” and “ApoER2”, as used herein, refers to any native LRP8 from any vertebrate source, including mammals such as primates (e.g. humans) and rodents (e.g., mice and rats), unless otherwise indicated. The term encompasses “full-length,” unprocessed LRP8 as well as any form of LRP8 that results from processing in the cell. The term also encompasses naturally occurring variants of LRP8, e.g., splice variants or allelic variants. The amino acid sequence of an exemplary human LRP8 is shown in SEQ ID NO:1: MGLPEPGPLRLLALLLLLLLLLLLRLQHLAAAAADPLLGG QGPAKECEKDQFQCRNERCIPSVWRCDEDDDCLDHSDEDDCPKKTCADSDFTCDNGH CIHERWKCDGEEECPDGSDESEATCTKQVCPAE [symbol] EGGADEAGCATLCAPHEFQCGNRSCLAAVFVCDGDDDCGDGSDERGCADPACGPRE FRCGGDGGGACIPERWVCDRQFDCEDRSDEAAELCGRPGPGATSAPAACATVSQFAC RSGECVHLGWRCDGDRDCKDKSDEADCPLGTCRGDEFQCGDGTCVLAIKHCNQEQD CPDGSDEAGCLQGLNECLHNNGGCSHICTDLKIGFECTCPAGFQLLDQKTCGDIDECK DPDACSQICVNYKGYFKCECYPGYEMDLLTKNCKAAGGKSPSLIFTNRYEVRRIDLVK RNYSRLIPMLKNVVALDVEVATNRIYWCDLSYRKIYSAYMDKASDPKEQEVLIDEQLH SPEGLAVDWVHKHIYWTDSGNKTISVATVDGGRRRTLFSRNLSEPRAIAVDPLRGFMY WSDWGDQAKIEKSGLNGVDRQTLVSDNIEWPNGITLDLLSQRLYWVDSKLHQLSSIDF SGGNRKTLISSTDFLSHPFGIAVFEDKVFWTDLENEAIFSANRLNGLEISILAENLNNPHD IVIFHELKQPRAPDACELSVQPNGGCEYLCLPAPQISSHSPKYTCACPDTMWLGPDMKR CYRAPQSTSTTTLASTMTRTVPATTRAPGTTVHRSTYQNHSTETPSLTAAVPSSVSVPR APSISPSTLSPATSNHSQHYANEDSKMGSTVTAAVIGIIVPIVVIALLCMSGYLIWRNWK RKNTKSMNFDNPVYRKTTEEEDEDELHIGRTAQIGHVYPAAISSFDRPLWAEPCLGETR EPEDPAPALKELFVLPGEPRSQLHQLPKNPLSELPVVKSKRVALSLEDDGLP (SEQ ID NO: 1) (Genbank BAA21824.1). In certain embodiments, the first 32 amino acids of SEQ ID NO: 1 are a signal sequence, and the sequence of an exemplary processed human LRP8 protein is given in SEQ ID NO: 2:AADPLLGGQGPAKEC EKDQFQCRNERCIPSVWRCDEDDDCLDHSDEDDCPKKTCADSDFTCDNGHCIHERWK CDGEEECPDGSDESEATCTKQVCPAEKLSCGPTSHKCVPASWRCDGEKDCEGGADEA GCATLCAPHEFQCGNRSCLAAVFVCDGDDDCGDGSDERGCADPACGPREFRCGGDG GGACIPERWVCDRQFDCEDRSDEAAELCGRPGPGATSAPAACATVSQFACRSGECVH LGWRCDGDRDCKDKSDEADCPLGTCRGDEFQCGDGTCVLAIKHCNQEQDCPDGSDE AGCLQGLNECLHNNGGCSHICTDLKIGFECTCPAGFQLLDQKTCGDIDECKDPDACSQI CVNYKGYFKCECYPGYEMDLLTKNCKAAGGKSPSLIFTNRYEVRRIDLVKRNYSRLIP MLKNVVALDVEVATNRIYWCDLSYRKIYSAYMDKASDPKEQEVLIDEQLHSPEGLAV DWVHKHIYWTDSGNKTISVATVDGGRRRTLFSRNLSEPRAIAVDPLRGFMYWSDWGD QAKIEKSGLNGVDRQTLVSDNIEWPNGITLDLLSQRLYWVDSKLHQLSSIDFSGGNRKT LISSTDFLSHPFGIAVFEDKVFWTDLENEAIFSANRLNGLEISILAENLNNPHDIVIFHELK QPRAPDACELSVQPNGGCEYLCLPAPQISSHSPKYTCACPDTMWLGPDMKRCYRAPQ STSTTTLASTMTRTVPATTRAPGTTVHRSTYQNHSTETPSLTAAVPSSVSVPRAPSISPS TLSPATSNHSQHYANEDSKMGSTVTAAVIGIIVPIVVIALLCMSGYLIWRNWKRKNTKS MNFDNPVYRKTTEEEDEDELHIGRTAQIGHVYPAAISSFDRPLWAEPCLGETREPEDPA PALKELFVLPGEPRSQLHQLPKNPLSELPVVKSKRVALSLEDDGLP (SEQ ID NO: 2).

An “LRP8-binding molecule” refers to a mole [symbol] LRP8-binding molecules include, but are not limited to, natural ligands of LRP8 (i.e., reelin, ApoE, or selenoprotein P), modified forms of natural ligands of LRP8, antibodies to LRP8, peptides that specifically bind to LRP8, aptamers that specifically bind to LRP8, and small molecules that specifically bind to LRP8 and LRP8-binding fragments of any of the foregoing. In certain embodiments, an LRP8-binding molecule does not compete for binding to LRP8 with a natural ligand of LRP8. In certain embodiments, an LRP8-binding molecule competes for binding to LRP8 with a natural ligand of LRP8. In certain embodiments, an LRP8-binding molecule antagonizes normal LRP8 function. In certain embodiments, an LRP8-binding molecule agonizes normal LRP8 function.

The term “modified form of a natural ligand of LRP8” refers to a natural ligand of LRP8 that has been modified such that it is no longer identical to the naturally-occurring form of the ligand of LRP8 yet still retains the ability to bind to LRP8. Such modification includes, but is not limited to, posttranslational modifications (i.e., glycosylation), amino acid modifications (i.e., substitutions, deletions or additions of amino acids, provided that the modified molecule retains binding to LRP8), fusions (i.e., Fc-fusions), and chemical modifications (i.e., attachment of a radiolabel or other detectable tag such as, but not limited to, a fluorophore or a hexahistidine tag).

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that i [symbol] moiety (e.g., a CNS-active compound) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

A “natural ligand of LRP8” or an “LRP8 natural ligand” refers to a polypeptide that is a naturally-occurring ligand of LRP8. Such naturally-occurring ligands include, but are not limited to, reelin, ApoE, selenoprotein P, and RAP. In certain embodiments, a natural ligand of LRP8 binds anywhere on LRP8. In certain embodiments, a natural ligand of LRP8 binds to an extracellular domain of LRP8. In certain embodiments, an LRP8-binding fragment of a natural ligand of LRP8 is used. The amino acid sequence of naturally occurring human reelin is:

MERSGWARQTFLLALLLGATLRARAAAGYYPRFSPFFFLCTHHGELEGDGEQGEVLIS LH IAGNPTYYVPGQEYHVTISTSTFFDGLLVTGLYTSTSVQASQSIGGSSAFGFGIMSDHQF GNQFMCSVVASHVSHLPTTNLSFIWIAPPAGTGCVNFMATATHRGQVIFKDALAQQLC EQGAPTDVTVHPHLAEIHSDSIILRDDFDSYHQLQLNPNIWVECNNCETGEQCGAIMHG NAVTFCEPYGPRELITTGLNTTTASVLQFSIGSGSCRFSYSDPSIIVLYAKNNSADWIQLE KIRAPSNVSTIIHILYLPEDAKGENVQFQWKQENLRVGEVYEACWALDNILIINSAHRQ VVLEDSLDPVDTGNWLFFPGATVKHSCQSDGNSIYFHGNEGSEFNFATTRDVDLSTEDI QEQWSEEFESQPTGWDVLGAVIGTECGTIESGLSMVFLKDGERKLCTPSMDTTGYGNL RFYFVMGGICDPGNSHENDIILYAKIEGRKEHITLDTLSYSSYKVPSLVSVVINPELQTPA TKFCLRQKNHQGHNRNVWAVDFFHVLPVLPSTMSHMIQFSINLGCGTHQPGNSVSLEF STNHGRSWSLLHTECLPEICAGPHLPHSTVYSSENYSGWNRITIPLPNAALTRNTRIRWR QTGPILGNMWAIDNVYIGPSCLKFCSGRGQCTRHGCKCDPGFSGPACEMASQTFPMFIS ESFGSSRLSSYHNFYSIRGAEVSFGCGVLASGKALVFNKEGRRQLITSFLDSSQSRFLQF TLRLGSKSVLSTCRAPDQPGEGVLLHYSYDNGIT [symbol] AKQFGIQFRWWQPYHSSQREDVWAIDEIIMTSVLFNSISLDFTNLVEVTQSLGFYLGNV QPYCGHDWTLCFTGDSKLASSMRYVETQSMQIGASYMIQFSLVMGCGQKYTPHMDN QVKLEYSTNHGLTWHLVQEECLPSMPSCQEFTSASIYHASEFTQWRRVIVLLPQKTWS SATRFRWSQSYYTAQDEWALDSIYIGQQCPNMCSGHGSCDHGICRCDQGYQGTECHP EAALPSTIMSDFENQNGWESDWQEVIGGEIVKPEQGCGVISSGSSLYFSKAGKRQLVS WDLDTSWVDFVQFYIQIGGESASCNKPDSREEGVLLQYSNNGGIQWHLLAEMYFSDFS KPRFVYLELPAAAKTPCTRFRWWQPVFSGEDYDQWAVDDIIILSEKQKQIIPVINPTLPQ NFYEKPAFDYPMNQMSVWLMLANEGMVKNETFCAATPSAMIFGKSDGDRFAVTRDL TLKPGYVLQFKLNIGCANQFSSTAPVLLQYSHDAGMSWFLVKEGCYPASAGKGCEGN SRELSEPTMYHTGDFEEWTRITIVIPRSLASSKTRFRWIQESSSQKNVPPFGLDGVYISEP CPSYCSGHGDCISGVCFCDLGYTAAQGTCVSNVPNHNEMFDRFEGKLSPLWYKITGA QVGTGCGTLNDGKSLYFNGPGKREARTVPLDTRNIRLVQFYIQIGSKTSGITCIKPRTRN EGLIVQYSNDNGILWHLLRELDFMSFLEPQIISIDLPQDAKTPATAFRWWQPQHGKHSA QWALDDVLIGMNDSSQTGFQDKFDGSIDLQANWYRIQGGQVDIDCLSMDTALIFTENI GKPRYAETWDFHVSASTFLQFEMSMGCSKPFSNSHSVQLQYSLNNGKDWHLVTEECV PPTIGCLHYTESSIYTSERFQNWKRITVYLPLSTISPRTRFRWIQANYTVGADSWAIDNV VLASGCPWMCSGRGICDAGRCVCDRGFGGPYCVPVVPLPSILKDDFNGNLHPDLWPE VYGAERGNLNGETIKSGTSLIFKGEGLRMLISRDLDCTNTMYVQFSLRFIAKSTPERSHS ILLQFSISGGITWHLMDEFYFPQTTNILFINVPLPYTAQTNATRFRLWQPYNNGKKEEIW IVDDFIIDGNNVNNPVMLLDTFDFGPREDNWFFYPGGNIGLYCPYSSKGAPEEDSAMVF VSNEVGEHSITTRDLNVNENTIIQFEINVGCSTDSSSADPVRLEFSRDFGATWHLLLPLC YHSSSHVSSLCSTEHHPSSTYYAGTMQGWRREVVHFGKLHLCGSVRFRWYQGFYPAG SQPVTWAIDNVYIGPQCEEMCNGQGSCINGTKCICDPGYSGPTCKISTKNPDFLKDDFE GQLESDRFLLMSGGKPSRKCGILSSGNNLFFNEDGLRMLMTRDLDLSHARFVQFFMRL GCGKGVPDPRSQPVLLQYSLNGGLSWSLLQEFLFSNSSNVGRYIALEIPLKARSGSTRL RWWQPSENGHFYSPWVIDQILIGGNISGNTVLEDDFTTLDSRKWLLHPGGTKMPVCGS TGDALVFIEKASTRYVVSTDVAVNEDSFLQIDFAASCSVTDSCYAIELEYSVDLGLSWH PLVRDCLPTNVECSRYHLQRILVSDTFNKWTRITLPLPPYTRSQATRFRWHQPAPFDKQ QTWAIDNVYIGDGCIDMCSGHGRCIQGNCVCDEQWGGLYCDDPETSLPTQLKDNFNR APSSQNWLTVNGGKLSTVCGAVASGMALHFSGGCSRLLVTVDLNLTNAEFIQFYFMY GCLITPNNRNQGVLLEYSVNGGITWNLLMEIFYDQYSKPGFVNILLPPDAKEIATRFRW WQPRHDGLDQNDWAIDNVLISGSADQRTVMLDTFSSAPVPQHERSPADAGPVGRIAF DMFMEDKTSVNEHWLFHDDCTVERFCDSPDGVMLCGSHDGREVYAVTHDLTPTEG WIMQFKISVGCKVSEKIAQNQIHVQYSTDFGVS [symbol] FPTKGWKRITYPLPESLVGNPVRFRFYQKYSDMQWAIDNFYLGPGCLDNCRGHGDCL REQCICDPGYSGPNCYLTHTLKTFLKERFDSEEIKPDLWMSLEGGSTCTECGILAEDTAL YFGGSTVRQAVTQDLDLRGAKFLQYWGRIGSENNMTSCHRPICRKEGVLLDYSTDGGI TWTLLHEMDYQKYISVRHDYILLPEDALTNTTRLRWWQPFVISNGIVVSGVERAQWA LDNILIGGAEINPSQLVDTFDDEGTSHEENWSFYPNAVRTAGFCGNPSFHLYWPNKKK DKTHNALSSRELIIQPGYMMQFKIVVGCEATSCGDLHSVMLEYTKDARSDSWQLVQT QCLPSSSNSIGCSPFQFHEATIYNSVNSSSWKRITIQLPDHVSSSATQFRWIQKGEETEKQ SWAIDHVYIGEACPKLCSGHGYCTTGAICICDESFQGDDCSVFSHDLPSYIKDNFESAR VTEANWETIQGGVIGSGCGQLAPYAHGDSLYFNGCQIRQAATKPLDLTRASKIMFVLQI GSMSQTDSCNSDLSGPHAVDKAVLLQYSVNNGITWHVIAQHQPKDFTQAQRVSYNVP LEARMKGVLLRWWQPRHNGTGHDQWALDHVEVVLVSTRKQNYMMNFSRQHGLRH FYNRRRRSLRRYP (Genbank AAC51105.1) (SEQ ID NO: 3). The amino acid sequence of naturally occurring human selenoprotein P is:

(SEQ ID NO: 4)
MWRSLGLALALCLLPSGGTESQDQSSLCKQPPAWSIRDQDPMLNSNGS
VTVVALLQASXYLCIIEASKLEDLRVKLKKEGYSNISYIVVNHQGISS
RLKYTHLKNKVSEHIPVYQQEENQTDVWTLLNGSKDDFLIYDRCGRLV
YHLGLPFSFLTFPYVEEAIKIAYCEKKCGNCSLTTLKDEDFCKRVSLA
TVDKTVETPSPHYHHEHHHNHGHQHLGSSELSENQQPGAPNAPTHPAP
PGLHHHHKHKGQHRQGHPENRDMPASEDLQDLQKKLCRKRCINQLLCK
LPTDSELAPRSXCCHCRHLIFEKTGSAITXQCKENLPSLCSXQGLRAE
ENITESCQXRLPPAAXQISQQLIPTEASASXRXKNQAKKXEXPSN.
(GenBank: CAA77836.1),

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment [symbol] being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In some embodiments, an agent is “peripherally administered” or “administered peripherally.” As used herein, these terms refer to any form of administration of an agent, e.g., a therapeutic agent, to an individual that is not direct administration to the CNS, e.g., that brings the agent in contact with the non-brain side of the blood-brain barrier. Peripheral administration includes, but is not limited to, intravenous, subcutaneous, intramuscular, intraperitoneal, transdermal, inhalational, transbuccal, intranasal, rectal and oral administration.

The term “pharmaceutical formulation” refers to a preparation which is in such form as to permit the biological activity of an active ingredient contained therein to be effective, and which contains no additional components which are the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “shuttle agent,” as used herein, refers to a compound comprising at least one LRP8-binding molecule and at least one CNS-active compound, wherein the at least one LRP8-binding molecule is conjugated to the at least one CNS-active compound either covalently (i.e., direct fusion or linker-mediated conjugation) or non-covalently (i.e., through ionic or hydrophobic interaction). Shuttle agents include, as a nonlimiting example, a multispecific fusion protein which specifically binds to each of LRP8 and a desired therapeutic or diagnostic CNS target.

A “therapeutic effect,” refers to the production of a condition that is better than the average or normal condition in an individual that is not suffering from a disorder (i.e., a supranormal effect such as improved cognition, memory, mood or other characteristic in a subject attributable at least in part to the functioning of the CNS, as compared to the normal or average state in an unafflicted or asymptomatic subject).

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, compositions and methods of the invention are used to delay development of a disease or disorder or to slow the progression of a disease or disorder (i.e., a CNS disease or a CNS disorder).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain maybe sufficient to confer antigen-binding specificity. Furthermore, antib [symbol] be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### II. Compositions and Methods

The blood-brain barrier (“BBB”) is a restrictive factor in the delivery of many peripherally-administered compounds to the central nervous system (CNS). In particular, the BBB places size restrictions on the molecules that can freely penetrate into the CNS. In general, large molecule therapeutics such as recombinant proteins, antisense drugs, gene medicines, monoclonal antibodies, or RNA interference (RNAi)-based drugs do not efficiently cross the BBB in pharmacologically significant amounts. While it is generally assumed that small molecule drugs can cross the BBB, in fact <2% of all small molecule drugs are active in the brain owing to their lack of transport across the BBB. Generally speaking, a molecule must be lipid soluble and have a molecular weight of less than 400 Daltons in order to cross the BBB in pharmacologically significant amounts, and the vast majority of small organic molecules do not possess both molecular characteristics. As such, the BBB is a limiting step in the development of new therapeutics, diagnostics and research tools for the brain and CNS.

To bypass or circumvent this BBB issue, invasive transcranial drug delivery strategies have been used, such as intracerebro-ventricular (ICV) infusion, intracerebral (IC) administration, and convection enhanced diffusion (CED). Implantable devices have also been used. Each of these methods of transcranial drug delivery to the brain are expensive, invasive and largely ineffective. The ICV route typically only delivers proteins to the ependymal surface of the brain, not into the brain parenchyma. Further, IC administration often results in delivery of a drug to only the localized area of the injection site due to the low efficiency of drug diffusion within the brain. The present invention offers an alternative to these highly invasive and generally unsatisfactory methods for bypassing the BBB, allowing agents, e.g., peptides, proteins and antibodies to cross the BBB from the peripheral blood. The invention is based on the use of an endogenous transport system p [symbol] mechanism to transport a desired substance from the peripheral blood to the CNS.

The present invention also addresses a key factor important in delivering an agent across the BBB to the CNS that has not yet been solved: brain-specific delivery to the brain. No highly brain-specific receptor-mediated delivery has yet been described. Most receptors previously utilized for delivery are also highly expressed in peripheral tissues, making solely brain-specific delivery impossible with those molecules. The advantages of improved brain-specificity are higher efficacy of the administered compound, as well as fewer potential side effects at a given dose of the compound. The present invention provides the surprising finding that LRP8 is a highly BBB-specific protein that can be exploited to facilitate translocation of other molecules normally unable to penetrate into the brain across the BBB, including biologic molecules such as antibodies.

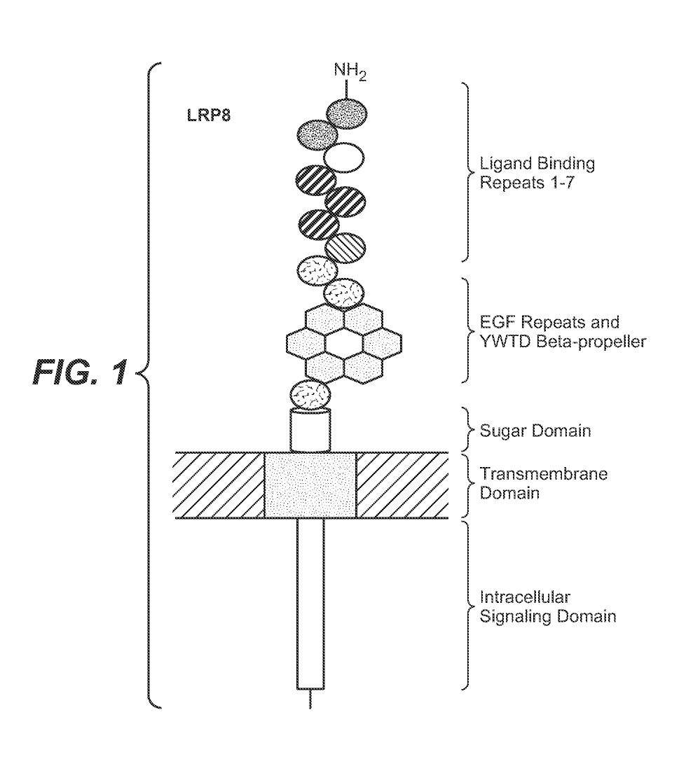

LRP8 is a member of the low density lipoprotein receptor (LDLR) family (Brown and Goldstein, Science 232 (1986): 34-47); however, LRP8 contains an additional exon that encodes a novel 59 amino acid segment in the cytoplasmic tail, including three potential copies of the minimal consensus sequence for the Src homology 3 (SH3)-binding motif (Riddell et al., J. Lipid Res. 40 (1999):1925-1930). A schematic of the structure of LRP8 is shown in FIG. 1. LRP8 has several extracellular domains including a ligand-binding repeat region near the N-terminus consisting of seven repeats (numbered consecutively, starting with 1 at the most N-terminal portion and 7 at the most C-terminal portion of the repeat region), an EGF repeat region, a YWTD beta-propeller region, and a sugar domain. After a short transmembrane region, the C-terminal signaling domain of the protein is located intracellularly.

LRP8 is much more specifically expressed in the brain/blood-brain barrier than other described transporters. It has previously been shown that in humans LRP8 is predominantly expressed in the brain and placenta and is undetectable in other tissues (Novak et al., J. Biol. Chem. 271: 11732-11736 (1996); Kim et al., J. Biol. Chem. 271: 8373-8380 (1996)). For therapeutics exploiting it, the high specificity of LRP8 not only permits utilization of a smaller dose of the therapeutic than would otherwise be necessary if binding also occurred in the periphery as well as at the brain, but it also may decrease or prevent potential side effects of a therapeutic by lowering the exposure of the therapeutic to other organs aside from the brain/CNS.

It is an object of the invention to provide therapeutic and diagnostic compositions capable of crossing the BBB and entering the brain to exert their therapeutic and/or diagnostic functions. Accordingly, the invention provides compounds comprising both an LRP8-binding molecule and a CNS-active compound, where the LR [symbol] effects transmigration of the compound across the BBB and the CNS-active compound has a therapeutic and/or diagnostic function desirable in the brain.

In one embodiment, the LRP8-binding molecule portion of the shuttle agent specifically binds to LRP8. In one aspect, the LRP8-binding molecule specifically binds to LRP8 in a manner that results in its translocation across the BBB. In one aspect, the LRP8-binding molecule specifically binds to LRP8 in a manner that results in the translocation of the LRP8-binding molecule and the remainder of its associated shuttle agent across the BBB.

In one embodiment, the LRP8-binding molecule portion of a shuttle agent of the invention is a natural ligand of LRP8. In one aspect, the LRP8-binding molecule is reelin. In one aspect, the LRP8-binding molecule is a protein having the amino acid sequence of SEQ ID NO:3. In one aspect, the LRP8-binding molecule is selenoprotein P. In one aspect, the LRP8-binding molecule is a protein having the amino acid sequence of SEQ ID NO: 4. In one embodiment, the LRP8-binding molecule portion of a shuttle agent of the invention is a modified form of a natural ligand of LRP8. In one aspect, the natural ligand is modified by the addition of a label. In one aspect, the label is selected from a radiolabel, a fluorophore, a chromophore, and an affinity tag. In one aspect, the natural ligand is modified by the addition, deletion, or substitution of one or more amino acids. In one aspect, the addition, deletion, or substitution modulates the binding of the modified natural ligand to LRP8 relative to the binding of the unmodified natural ligand to LRP8. In one such aspect, the modulation is increased binding. In one such aspect, the modulation is decreased binding. In one aspect, the addition, deletion or substitution modulates the translocation of the modified natural ligand by LRP8 across the BBB. In one such aspect, the translocation across the BBB is increased. In one such aspect, the translocation across the BBB is decreased. In one aspect, the modified form of a natural ligand is a modified reelin. In one aspect, the modified form of a natural ligand is a modified protein of SEQ ID NO: 3. In one aspect, the modified form of a natural ligand is a modified selenoprotein P. In one aspect, the modified form of a natural ligand is a modified protein of SEQ ID NO: 4. In one aspect, the modified form of a natural ligand is an immunoconjugate.

In one embodiment, the LRP8-binding molecule portion of a shuttle agent of the invention is an LRP8-binding fragment of a natural ligand of LRP8. In one aspect, the LRP8-binding fragment is a fragment of reelin. In one aspect, the LRP8-binding fragment is a fragment of a protein having the amino acid sequence of SEQ ID NO:3. In one aspect, the LRP8-binding fragment is a fragment of selenoprotein P. In one aspect, the LRP8-binding fragment is a fragment of a protein having the amino a [symbol] embodiment, the LRP8-binding molecule portion of a shuttle agent of the invention is an LRP8-binding fragment of a modified form of a natural ligand of LRP8. In one aspect, the natural ligand is modified by the addition of a label. In one aspect, the label is selected from a radiolabel, a fluorophore, a chromophore, and an affinity tag. In one aspect, the natural ligand is modified by the addition, deletion, or substitution of one or more amino acids. In one aspect, the addition, deletion, or substitution modulates the binding of the LRP8-binding fragment of the modified natural ligand to LRP8 relative to the binding of the unmodified LRP8-binding fragment of the natural ligand to LRP8. In one such aspect, the modulation is increased binding. In one such aspect, the modulation is decreased binding. In one aspect, the addition, deletion or substitution modulates the translocation of the LRP8-binding fragment of the modified natural ligand by LRP8 across the BBB. In one such aspect, the translocation across the BBB is increased. In one such aspect, the translocation across the BBB is decreased. In one aspect, the modified form of an LRP8-binding fragment of a natural ligand is an LRP8-binding fragment of modified reelin. In one aspect, the modified form of an LRP8-binding fragment of a natural ligand is an LRP8-binding fragment of a modified protein of SEQ ID NO: 3. In one aspect, the modified form of an LRP8-binding fragment of a natural ligand is an LRP8-binding fragment of a modified selenoprotein P. In one aspect, the modified form of an LRP8-binding fragment of a natural ligand is an LRP8-binding fragment of a modified protein of SEQ ID NO: 4. In one aspect, the modified form of an LRP8-binding fragment of a natural ligand is an immunoconjugate.

In one embodiment, the LRP8-binding molecule portion of a shuttle agent of the invention binds to the ligand binding repeat region of LRP8. In one embodiment, the LRP8-binding molecule portion of a shuttle agent of the invention binds to ligand binding repeat 1 of LRP8. In one embodiment, the LRP8-binding molecule portion of a shuttle agent of the invention binds to ligand binding repeat 2 of LRP8. In one embodiment, the LRP8-binding molecule portion of a shuttle agent of the invention binds to ligand binding repeat 3 of LRP8. In one embodiment, the LRP8-binding molecule portion of a shuttle agent of the invention binds to ligand binding repeat 4 of LRP8. In one embodiment, the LRP8-binding molecule portion of a shuttle agent of the invention binds to ligand binding repeat 5 of LRP8. In one embodiment, the LRP8-binding molecule portion of a shuttle agent of the invention binds to ligand binding repeat 6 of LRP8. In one embodiment, the LRP8-binding molecule portion of a shuttle agent of the invention binds to ligand binding repeat 7 of LRP8. In one embodiment, the LRP8-binding molecule portion of a shuttle agent of the invention binds to the EGF repeat region of LRP8. In one embodiment, the LRP8-bindi [symbol] the invention binds to the YWTD beta-propeller region of LRP8. In one embodiment, the LRP8-binding molecule portion of a shuttle agent of the invention binds to the sugar domain region of LRP8.

In one embodiment, the LRP8-binding molecule portion of a shuttle agent is an antibody. In one aspect, the antibody is a monoclonal antibody. In one aspect, the antibody is a chimeric antibody. In one aspect, the antibody is a humanized antibody. In one aspect, the antibody is a human antibody. In one aspect, the antibody is a multispecific antibody. In one aspect, the antibody is labeled. In one such aspect, the label is selected from a radiolabel, a fluorophore, a chromophore and an affinity tag. In one aspect, the antibody has an affinity for LRP8 sufficient to permit its translocation across the BBB. In one aspect, the antibody has an affinity for LRP8 sufficient to permit the translocation of the remainder of the shuttle agent of which it is a part across the BBB. In one aspect, the antibody does not have effector function. In one aspect, the antibody does have effector function. In one aspect, the antibody isotype is selected from IgGl, IgG2, IgG3, IgG4, IgM, IgA, IgD and IgE. In one aspect, the antibody binds to the extracellular domain of LRP8. In one aspect, the antibody competes for binding to the extracellular domain of LRP8 with one or more natural ligands of LRP8. In one aspect, the antibody does not compete for binding to the extracellular domain of LRP8 with one or more natural ligands of LRP8. In another aspect, the antibody of any of the foregoing aspects is an antibody fragment that binds LRP8.

In one embodiment, the LRP8-binding molecule portion of a shuttle agent is selected from a peptide, an aptamer, and a small molecule. In one aspect, the binding affinity of the LRP8-binding molecule is sufficient to permit its translocation across the BBB. In one aspect, the LRP8-binding molecule has an affinity for LRP8 sufficient to permit the translocation of the remainder of the shuttle agent of which it is a part across the BBB. In one aspect, the LRP8-binding molecule is an LRP8-binding peptide. In one aspect, the LRP8-binding molecule is an aptamer. In one aspect, the LRP8-binding molecule is a small molecule.

In one embodiment, the CNS-active compound portion of a shuttle agent is a compound with an effect on one or more CNS targets. In one aspect, the effect is useful in research. In one aspect, the effect is a therapeutic effect. In one aspect, the effect is an effect useful in diagnostics. In one such aspect, the CNS-active compound binds to one or more targets in the CNS and permits imaging of those targets. In one such aspect, the binding of the CNS-active compound to one or more CNS targets is indicative of the presence or progression of a CNS disease or disorder.

In one embodiment, the CNS-active compoun [symbol] its ability to treat a CNS disease or disorder. In one aspect, the CNS disease or disorder is a neuropathy. In one aspect, the CNS disease or disorder is an amyloidosis. In one aspect, the CNS disease or disorder is a cancer. In one aspect, the CNS disease or disorder is an ocular disease or disorder. In one aspect, the CNS disease or disorder is a viral or microbial infection. In one aspect, the CNS disease or disorder is inflammation. In one aspect, the CNS disease or disorder is ischemia. In one aspect, the CNS disease or disorder is a neurodegenerative disease. In one aspect, the CNS disease or disorder is a seizure. In one aspect, the CNS disease or disorder is a behavioral disorder. In one aspect, the CNS disease or disorder is a lysosomal storage disease. In one aspect, the CNS-active compound eliminates the disease or disorder. In one aspect, the CNS-active compound lessens the severity or duration of the disease or disorder. In one aspect, the CNS-active compound prevents the onset or progression of the disease or disorder.

In one embodiment, the CNS-active compound portion of a shuttle agent is selected for its ability to detect a CNS disease or disorder. In one aspect, the CNS disease or disorder is a neuropathy. In one aspect, the CNS disease or disorder is an amyloidosis. In one aspect, the CNS disease or disorder is a cancer. In one aspect, the CNS disease or disorder is an ocular disease or disorder. In one aspect, the CNS disease or disorder is a viral or microbial infection. In one aspect, the CNS disease or disorder is inflammation. In one aspect, the CNS disease or disorder is ischemia. In one aspect, the CNS disease or disorder is a neurodegenerative disease. In one aspect, the CNS disease or disorder is a seizure. In one aspect, the CNS disease or disorder is a behavioral disorder. In one aspect, the CNS disease or disorder is a lysosomal storage disease. In one aspect, the CNS-active compound detects the disease or disorder before the onset of symptoms. In one aspect, the CNS-active compound permits assessment of the severity or duration of the disease or disorder. In one aspect, the CNS-active compound permits noninvasive detection and/or imaging of the disease or disorder. In one aspect, the imaging is by radiography. In one aspect, the imaging is by tomography. In one aspect, the imaging is by magnetic resonance imaging.

In one embodiment, a CNS-active compound is selected from an antibody, a protein, a peptide, an aptamer, an inhibitory nucleic acid, and a small molecule, or a fragment of any of the foregoing. In one aspect, the CNS-active compound is an antibody. In one aspect, the CNS-active compound is a protein. In one aspect, the CNS-active compound is a peptide. In one aspect, the CNS-active compound is an aptamer. In one aspect, the CNS-active compound is an inhibitory nucleic acid. In one such aspect, the inhibitory nucleic acid is an siRNA. In one such aspect, the inhibitory nucleic acid is a ribozy [symbol] compound is a small molecule. In one aspect, a CNS-active compound is a fragment of any of the foregoing which retains its activity in the CNS. In one aspect, a CNS-active compound is labeled. In one such aspect, the label is selected from a radiolabel, a fluorophore, a chromophore and an affinity tag.

In one embodiment, the LRP8-binding molecule and the CNS-active compound are conjugated. In one aspect, the conjugation is by direct conjugation without a linker. In one aspect, the conjugation is by means of a linker moiety. In one aspect, the conjugation is covalent. In one aspect, the conjugation is noncovalent. In one aspect, the linker is cleavable within the CNS. In one aspect, the conjugation results in each of the LRP8-binding molecule and the CNS-active compound retaining a proportion of their normal binding and/or activity. In one such aspect, 100% of the original activity of each is retained.

In one embodiment, the LRP8-binding molecule is a multispecific antibody. In one aspect the multispecific antibody is bispecific. In one such aspect, the antibody specifically binds to LRP8 and also binds to another CNS target by means of the CNS-binding compound which contributes the second specificity.

In one embodiment, a shuttle agent comprises more than one LRP8-binding molecule. In one aspect, each instance of the LRP8-binding molecule is the same. In one aspect, the LRP8-binding molecules differ. In one embodiment, a shuttle agent comprises more than one CNS-active compound. In one aspect, each instance of the CNS-active compound in the shuttle agent is the same. In one aspect, the CNS-active compounds differ.

In one embodiment, a shuttle agent of the invention is modified to modulate the serum half-life of the molecule. In one aspect, the LRP8-binding molecule portion of the shuttle agent is modified. In one aspect, the CNS-active compound portion of the shuttle agent is modified. In one aspect, both the LRP8-binding molecule portion and the CNS-active compound portions of the shuttle agent are modified. In one aspect, the modification is glycosylation. In one aspect, the modification is pegylation. In one aspect, the modification is the addition of an Fc domain. In certain aspects, the serum half-life is an average of at least about 5-fold greater than that of the unmodified shuttle agent. In one embodiment, any of the foregoing compounds of the invention are formulated.

In one aspect, the formulation is sterile. In one aspect, the formulation is with a pharmaceutically acceptable carrier. In one aspect, the formulation is appropriate for the route of administration. In one aspect, the formulation further comprises an additional compound. In one such aspect, the additional compound is selected to treat the desired

In one embodiment, nucleic acid molecule(s) e [symbol] shuttle agent are provided. In one aspect, a nucleic acid molecule encoding the LRP8-binding molecule portion of a shuttle agent is provided. In one aspect, a nucleic acid molecule encoding the CNS-active compound portion of a shuttle agent is provided. In one aspect, a single nucleic acid molecule encoding both the LRP8-binding molecule and CNS-active compound portions of a shuttle agent is provided. In one embodiment, a vector encompassing any of the foregoing nucleic acid molecules is provided. In one aspect the vector is an expression vector. In one embodiment, a host cell transformed with a vector of the invention is provided. In one aspect the host cell is a prokaryotic host cell. In one aspect the host cell is a eukaryotic host cell. In one embodiment, the invention provides a method of making a shuttle agent of the invention. In one aspect, the shuttle agent is entirely proteinaceous, and is expressed and purified from a host cell of the invention. In one aspect, only a portion of the shuttle agent is proteinaceous, and that portion is expressed and purified from a host cell of the invention.

In one embodiment, any of the foregoing compositions of the invention are used for manufacturing a medicament. In one embodiment, any of the foregoing compositions of the invention are used for manufacturing a medicament for treatment of a CNS disease or disorder. In one aspect, the CNS disease or disorder is a neuropathy. In one aspect, the CNS disease or disorder is an amyloidosis. In one aspect, the CNS disease or disorder is a cancer. In one aspect, the CNS disease or disorder is an ocular disease or disorder. In one aspect, the CNS disease or disorder is a viral or microbial infection. In one aspect, the CNS disease or disorder is inflammation. In one aspect, the CNS disease or disorder is ischemia. In one aspect, the CNS disease or disorder is a neurodegenerative disease. In one aspect, the CNS disease or disorder is a seizure. In one aspect, the CNS disease or disorder is a behavioral disorder. In one aspect, the CNS disease or disorder is a lysosomal storage disease.

In one embodiment, any of the foregoing compositions of the invention are used for manufacturing a medicament for detection of a CNS disease or disorder. In one aspect, the CNS disease or disorder is a neuropathy. In one aspect, the CNS disease or disorder is an amyloidosis. In one aspect, the CNS disease or disorder is a cancer. In one aspect, the CNS disease or disorder is an ocular disease or disorder. In one aspect, the CNS disease or disorder is a viral or microbial infection. In one aspect, the CNS disease or disorder is inflammation. In one aspect, the CNS disease or disorder is ischemia. In one aspect, the CNS disease or disorder is a neurodegenerative disease. In one aspect, the CNS disease or disorder is a seizure. In one aspect, the CNS disease or disorder is a behavioral dis [symbol] disorder is a lysosomal storage disease.

In one embodiment methods of using the foregoing compositions of the invention to treat a CNS disease or disorder in a subject are provided. In one embodiment methods of using the foregoing compositions of the invention to detect, stage or monitor the progress of a

CNS disease or disorder in a subject are provided. In one aspect, any of the foregoing compositions are administered peripherally. In one aspect, the administration is oral. In one aspect, the administration is intravenous. In one aspect, the administration is intramuscular. In one aspect, the administration is subcutaneous. In one aspect, the administration is intraperitoneal. In one aspect, the administration is rectal. In one aspect, the administration is transbuccal. In one aspect, the administration is intranasal. In one aspect, the administration is transdermal. In one aspect, the administration is inhalational. In one aspect, about 1 mg to about 100 mg of the composition is administered.

In one embodiment, a CNS disease or disorder may be treated by administering a shuttle agent of the invention in conjunction with a noninvasive therapy. In one aspect the therapy is radiation treatment. In another aspect the therapy is behavioral therapy or psychotherapy.

A. Exemplary Shuttle Agents

In one aspect, the invention provides isolated shuttle agents that bind to LRP8 and translocate across the BBB into the CNS. As described herein, a shuttle agent comprises at least one LRP8-binding molecule and at least one CNS-active compound. The conjugation between the at least one LRP8-binding molecule and the at least one CNS-active compound

The LRP8-binding molecule portion of the shuttle agent specifically binds to LRP8 and facilitates the translocation of the shuttle agent across the BBB. The identification and preparation of binding molecules that specifically bind to a given antigen is well known in the art. Certain molecules which can be LRP8-binding molecules of the invention include, but are not limited to, antibodies, peptides, natural ligands of LRP8 (i.e., reelin, ApoE, or selenoprotein P), modified versions of natural ligands of LRP8, aptamers, and small molecules, and fragments of any of the foregoing that retain LRP8-binding activity. In certain embodiments, an LRP8-binding molecule does not compete for binding to LRP8 with a natural ligand of LRP8. In certain embodiments, an LRP8-binding molecule competes for binding to LRP8 with a natural ligand of LRP8. In certain embodiments, an LRP8-binding molecule does not interfere with the normal functioning of LRP8. In certain embodiments, an LRP8-binding molecule antagonizes normal LRP8 function. In certa [symbol] molecule agonizes normal LRP8 function.

The CNS-active compound portion of the shuttle agent has an effect within the CNS of a subject. CNS active compounds include, but are not limited to, therapeutic compounds, diagnostic compounds, and compounds with an effect useful in research. Therapeutic CNS-active compounds are compounds that are effective to treat one or more CNS diseases or disorders, to prevent the onset or development of one or more CNS diseases or disorders, or to decrease or prevent the severity, duration, or symptoms of one or more CNS diseases or disorders. Diagnostic CNS active compounds are compounds that are effective in diagnosing or staging one or more CNS diseases or disorders, or in imaging one or more areas of the brain.

Certain molecules which can be a CNS-active compound of the invention include, but are not limited to, a CNS target binding molecule, a CNS target inhibitor, a CNS target activator, a drug, an enzyme, a cyototoxin, and a detection molecule (general to the CNS or specific for a particular CNS target). Certain molecules which can be CNS-active compounds of the invention include, but are not limited to, antibodies, peptides, proteins, natural ligands of one or more CNS target(s), modified versions of natural ligands of one or more CNS target(s), aptamers, inhibitory nucleic acids (i.e., small inhibitory RNAs (siRNA) and short hairpin RNAs (shRNA)), ribozymes, and small molecules, or active fragments of any of the foregoing. Exemplary CNS-active compounds of the invention are described herein and include, but are not limited to, antibodies, aptamers, proteins, peptides, inhibitory nucleic acids and small molecules and active fragments of any of the foregoing that either are themselves or specifically recognize and/or act upon (i e , inhibit, activate, or detect) a CNS target molecule such as, but not limited to, amyloid precursor protein or portions thereof, amyloid beta, beta-secretase, gamma-secretase, tau, alpha-synuclein, parkin, huntingtin, DR6, presenilin, ApoE, glioma or other CNS cancer markers, and neurotrophins

When an antibody is conjugated to a drug, the resulting complex is termed an antibody-drug conjugate (ADC). In one embodiment, a shuttle agent is an ADC. In addition to previously known therapeutic compounds for treatment of a CNS disease or disorder described herein, e.g., in Section G below, which may be used as CNS-active compounds in certain embodiments, general cytotoxic drugs may be used as the one or more CNS-active compounds in a shuttle agent of the invention. Such drugs include, but are not limited to, a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,7 [symbol] 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, a shuttle agent comprises at least one CNS-active compound which is an enzymatically active toxin or fragment thereof Such toxins or fragments thereof include, but are not limited to, diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

In any of the foregoing embodiments, a shuttle agent may comprise one or more portions which incorporate a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

In any of the above embodiments, an antibody comprised in a shuttle agent may be humanized. In one such embodiment, an antibody comprised in a shuttle agent comprises hypervariable regions (HVRs) from a protein of a nonhuman species, and further comprises an acceptor human framework, e.g. a human immunoglobulin framework or a human consensus framework.

In a further aspect of the invention, an antibody comprised in a shuttle agent according to any of the above embodiments is a monoclonal antibody, including a chimeric, humanized or human antibody. In one embodiment, an antibody comprised in a shuttle agent is an antibody fragment, e.g., a Fv, Fab, Fab′, scFv, diabod [symbol] embodiment, the antibody is a full length antibody, e.g., an intact antibody of any antibody class or isotype as defined herein. In a further aspect, a shuttle agent according to any of the above embodiments may incorporate any of the features, singly or in combination, as described in Sections 1-7 below:
1. Affinity

In certain embodiments, a shuttle agent provided herein has sufficient affinity for LRP8 to permit LRP8-mediated translocation of the conjugated CNS-active compound. In certain embodiments, a shuttle agent provided herein has at least two affinities: the LRP8-binding molecule portion has an affinity for LRP8, and the CNS-active compound has an affinity for at least one CNS target. In certain embodiments, a shuttle agent provided herein has a dissociation constant (Kd) of ≦104, ≦100 nM, ≦10 nM, ≦1 nM, ≦0.1 nM, ≦0.01 nM, or ≦0.001 nM (e.g. 10⁻⁸M or less, e.g. from 10⁻⁸M to 10⁻¹³M, e.g., from 10⁻⁹M to 10⁻¹³ M).

In one embodiment, Kd is measured by a radiolabeled antigen binding assay (RIA) performed with a protein of interest and its target binding partner, according to standard techniques well known in the art. For example, for a shuttle agent comprising a Fab, the assay can be performed as follows. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody- coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrat [symbol] equal to 20% of maximal binding are chosen for use in competitive binding assays.

According to another embodiment, Kd is measured using surface plasmon resonance assays using a BIACORE®-2000 or a BIACORE®-3000 (BlAcore, Inc., Piscataway, N.J.) at 25° C. with immobilized target (i.e., LRP8, or a CNS target) CM5 chips at ˜10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Target is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of the protein of interest whose Kd is to be determined (for example, a Fab, antibody, immunoconjugate, or other binding protein) (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE ® Evaluation Software version 3.2) by simultaneously fitting the association and dissociation sensorgrams. The equilibrium dissociation constant (Kd) is calculated as the ratio k_{off}/k_{on.} See, e.g., Chen et al., J. Mol. Biol. 293:865-881 (1999). If the on-rate exceeds 10⁶ M⁻¹ 5⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette. As will be understood by one of ordinary skill in the art, the foregoing assay may be readily adapted to measurement of non-antibody binding molecules.
2. Fragments

In certain embodiments, the LRP8-binding molecule portion of a shuttle agent is an LRP8-binding fragment of a larger LRP8-binding protein. In certain embodiments, the LRP8-binding molecule portion of a shuttle agent is an LRP8-binding fragment of a natural ligand of LRP8, or a modified version thereof. In certain embodiments, the LRP8-binding molecule portion of a shuttle agent is an LRP8-binding fragment of an anti-LRP8 antibody. Antibody fragments include, but are not limited to, Fab, Fab′, F [symbol] and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

In certain embodiments, a shuttle agent comprises a fragment of a multispecific antibody. Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

In certain embodiments, a shuttle agent comprises one or more single-domain antibodies. Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Fragments can be made by various techniques, including but not limited to proteolytic digestion of an protein as well as production by recombinant host cells (e.g. E. coli or phage), as described herein.
3. Chimeric and Humanized Molecules

A shuttle agent herein comprises both an LRP8-binding molecule and a CNS-active compound, and as such is a chimeric molecule. In certain embodiments, the LRP8-binding molecule portion of a shuttle agent provided herein is a chimeric molecule. In certain embodiments, the CNS-active compound portion of a shuttle agent provided herein is a chimeric molecule. In certain embodiments, an antibody provided herein as a shuttle agent or a portion thereof is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been chan [symbol] Chimeric antibodies include antigen-binding fragments thereof.

It is understood by one of ordinary skill in the art that it may be desirable to modify one or more amino acids in a shuttle agent derived from a non-human species to make it more ‘human’ in appearance to a human immune system and/or to increase its activity or half-life when administered to a human. “Humanization” is most frequently performed in the context of a non-human antibody or a chimeric antibody, where none or some, respectively, of the antibody is derived from a human molecule. Shuttle agents comprising a non-human or chimeric antibody are humanized in certain embodiments. In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5, 821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR librarie [symbol] 272:10678-10684 (1997) and Rosok et al., J. Biol. Chem. 271:22611-22618 (1996)).
4. Human Molecules

In certain embodiments, a shuttle agent provided herein is human in origin. In certain embodiments, an antibody comprised in a shuttle agent provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HuMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Fin [symbol] Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.
5. Library-Derived Proteins

Shuttle agents, LRP8-binding molecules, and CNS-active compounds of the invention may be isolated by screening combinatorial libraries for shuttle agents, LRP8-binding molecules or CNS-active compounds with the desired activity or activities. For example, a variety of methods are known in the art for generating phage display libraries and screening such libraries for proteins possessing the desired binding characteristics. Such methods are reviewed, e.g., in Sidhu et al., eds., Phage Display In Biotechnology and Drug Discovery, CRC Press (2005); Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods directed to antibody identification, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: [symbol] Publication Nos. 2005/0079574, 2005/0119455, 2005/0266000, 2007/0117126, 2007/0160598, 2007/0237764, 2007/0292936, and 2009/0002360.

Proteins (i.e., antibodies or antibody fragments) isolated from human libraries are considered human proteins herein.
6. Multispecific Antibodies

In certain embodiments, a shuttle agent provided herein is a multispecific antibody, e.g. a bispecific antibody. Multispecific antibodies are monoclonal antibodies that have binding specificities for at least two different sites. In certain embodiments, one of the binding specificities is for LRP8 and the other is for any other CNS target. In certain embodiments, multispecific antibodies may bind to two different epitopes of LRP8 and any other CNS target. Multispecific antibodies may also be used to localize CNS-active compounds to cells which express LRP8. Multispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to LRP8 as well as another, different antigen (see, US 2008/0069820, for example), i.e., a CNS target.
7. Shuttle Agent Variants

In certain embodiments, amino acid sequence variants of the shuttle agents provided herein are contemplated. For example, it may be desirable to improve the binding affinity and/or other biological properties of either the LRP8-binding molecule portion or the CNS-active compound portion of the shuttle agent, or both, when such portions are proteins. Amino acid sequence variants of all or part of a shuttle agent may be prepared by introducing appropriate modifications into the nucleotide sequence encoding the shuttle agent or a portion thereof, or by peptide synthesis. Such modifications include, for example, deletions from, and/or insertions into and/or substitutions of residues within the amino acid sequences of the shuttle agent. Any combination of deletion, insertion, and substitution can be made to arrive at the final construct, provided that the final construct possesses the desired characteristics, e.g., target-binding, such as binding to LRP8 and binding to one or more desired CNS targets.
a) Substitution, Insertion, and Deletion Variants

In certain embodiments, shuttle agent variants having one or more amino acid substitutions are provided. Variations in the shuttle agents provided herein can be made, for example, using any of the techniques and guidelines for conservative and non-conservative mutations set forth, for instance, in U.S. Pat. No. 5,364,934. When the portion(s) of a shuttle agent under consideration for modification is an antibody, sites of interest for substitutional mutagenesis include the hypervariable regions and framework regions. Conservative amino acid substitutions are shown in Table 1 under the heading of “conservative substitutions.” More substantial changes are provided in Table 1 under the heading of “exemplary substitutions,” and as further described below in reference to amino acid side chain classes. Guidance in determining which amino acid residue(s) may be inserted, substituted or deleted without adversely affecting the desired activity may be found by comparing the sequence of the LRP8-binding molecule and/or the CNS-active compound of the shuttle agent with that of homologous known protein molecules and minimizing the number of amino acid sequence changes made in regions of high homology. Scanning amino acid analysis can also be employed to identify one or more amino acids along a contiguous sequence. Amino acid substitutions may be introduced into a shuttle agent of interest and the products screened for a desired activity, e.g., retained/improved binding to LRP8 and/or a CNS target or decreased immunogenicity=.

TABLE

[symbol]

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; | Leu |
| | Phe; Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Trp; Leu; Val; Ile; | Tyr |
| | Ala; Tyr | |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; | Leu |
| | Ala; Norleucine | |

[symbol]

indicates data missing or illegible when filed

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

One type of substitutional variant in an antibody context involves substituting one or more hypervariable region residues of a parent antibody (e.g. a humanized or human antibody). Generally, the resulting variant(s) selected for further [symbol] improvements) in certain biological properties (e.g., increased affinity, reduced immunogenicity) relative to the parent antibody and/or will have substantially retained certain biological properties of the parent antibody. An exemplary substitutional variant of this type is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O′Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine [symbol] interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.
b) Glycosylation Variants

In certain embodiments, a shuttle agent provided herein is altered to increase or decrease the extent to which the shuttle agent is glycosylated. Addition or deletion of glycosylation sites to a shuttle agent may be conveniently accomplished by altering the amino acid sequence such that one or more glycosylation sites is created or removed.

Where the shuttle agent comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (G1cNAc), galactose, and sialic acid, as well as a fucose attached to a GlcNAc in the “stem” of the biantennary oligosaccharide structure. In some embodiments, modifications of the oligosaccharide in a shuttle agent of the invention may be made in order to create shuttle agent variants with certain improved properties.

In one embodiment, shuttle agent variants are provided having a carbohydrate structure that lacks fucose attached (directly or indirectly) to an Fc region. For example, the amount of fucose in such shuttle agent may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose stru [symbol] mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated proteins include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Shuttle agent variants are further provided with bisected oligosaccharides, e.g., in which a biantennary oligosaccharide attached to the Fc region of the shuttle agent is bisected by GlcNAc. Such shuttle agent variants may have reduced fucosylation and/or improved ADCC function. Examples of such shuttle agent variants are described, e.g., in WO 2003/011878 (Jean-Mairet et al.); U.S. Pat. No. 6,602,684 (Umana et al.); and US 2005/0123546 (Umana et al.). Shuttle agent variants with at least one galactose residue in the oligosaccharide attached to an Fc region are also provided. Such variants may have improved CDC function. Such variants in the antibody context are described, e.g., in WO 1997/30087 (Patel et al.); WO 1998/58964 (Raju, S.); and WO 1999/22764 (Raju, S.).
c) Fc Region Variants

In certain embodiments, one or more amino acid modifications may be introduced into the Fc region comprised in a shuttle agent provided herein, thereby generating an Fc region variant. The Fc region variant may comprise a human Fc region sequence (e.g., a human IgG1, IgG2, IgG3 or IgG4 Fc region) comprising an amino a [symbol] one or more amino acid positions.

In certain embodiments, the invention contemplates a shuttle agent comprising an Fc variant that possesses some but not all effector functions, which make it a desirable candidate for applications in which the half life of the shuttle agent in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcyR binding (hence likely lacking ADCC activity), but retains FcRn binding ability. The primary cells for mediating ADCC, NK cells, express FcyRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Fc-containing shuttle agents with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, i [symbol] mutant with substitution of residues 265 and 297 to alanine (U.S. Pat. No. 7,332,581).

Certain Fc variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an Fc-containing shuttle agent variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in

US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. No. 5,648,260; U.S. Pat. No. 5,624,821; and WO 94/29351 concerning other examples of Fc region variants.
d) Cysteine Engineered Antibody Variants

In certain embodiments, when a shuttle agent comprises an antibody or an antibody fragment, it may be desirable to create cysteine engineered antibodies, e.g., “thioMAbs,” in which one or more residues of an antibody or antibody fragment are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as d [symbol] 7,521,541.
e) Derivatives

In certain embodiments, a shuttle agent provided herein may be further modified to contain additional nonproteinaceous moieties that are known in the art and readily available. The moieties suitable for derivatization of the shuttle agent include but are not limited to water soluble polymers. Non-limiting examples of water soluble polymers include polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the shuttle agent may vary, and if more than one polymer is attached, they can be the same or different molecules. In general, the number and/or type of polymers used for derivatization can be determined based on considerations including, but not limited to, the particular properties or functions of the shuttle agent to be improved, whether the shuttle agent derivative will be used in a therapy under defined conditions, etc.

In another embodiment, conjugates of a shuttle agent and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

B. Recombinant Methods and Compositions

Shuttle agents may be produced using recombinant methods and compositions which are well known in the art. For one example of production and purification of an antibody, see U.S. Pat. No. 4,816,567. In one embodiment, isolated nucleic acid encoding all or a portion of a shuttle agent described herein is provided. When the shuttle agent is a multispecific antibody, or where a portion of a shuttle agent is an antibody, such nucleic acid may encode an amino acid sequence comprising the VL and/or an am [symbol] the antibody (e.g., the light and/or heavy chains of the antibody).

In a further embodiment, one or more vectors (e.g., expression vectors) comprising such nucleic acid are provided. In a further embodiment, a host cell comprising such nucleic acid is provided. In one such embodiment where the shuttle agent or portion thereof is an antibody or antibody fragment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., YO, NSO, Sp20 cell).

In one embodiment, a method of making a shuttle agent or portion thereof is provided, wherein the method comprises culturing a host cell comprising a nucleic acid encoding the shuttle agent or portion thereof, as provided above, under conditions suitable for expression of the shuttle agent or portion thereof, and optionally recovering the antibody from the host cell (or host cell culture medium).

For recombinant production of a shuttle agent, nucleic acid encoding a shuttle agent, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to gene(s) encoding the shuttle agent).

Suitable host cells for cloning or expression of shuttle agent-encoding, LRP8-binding molecule-encoding, or CNS-active compound-encoding vectors include prokaryotic or eukaryotic cells described herein. For example, shuttle agents may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B.K.C. Lo, ed., Humana Press, Totowa, N.J., 2003), pp. 245-254, describing expression of antibody fragments in E. coli.) After expression, the shuttle agent may be isolated from the bacterial cell paste in a soluble fraction and can be further purified.

In addition to prokaryotes, eukaryotic microbes such as filamentous fungi or yeast are suitable cloning or expression hosts for shuttle agent-encoding vectors, including fungi and yeast strains whose glycosylation pathways have been “humanized,” resulting in the production of a shuttle agent with a partially or fully human glyco [symbol] Biotech. 22:1409-1414 (2004), and Li et al., Nat. Biotech. 24:210-215 (2006). Suitable prokaryotic and eukaryotic host cells for protein expression are described in, e.g., PCT publication no. WO2008080045.

Suitable host cells for the expression of glycosylated shuttle agent are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., Thomas et al., “Production of Therapeutic Products in Plants,” U. Calif. Agricultural Biotechnology in California Series, Pub. No. 8078 (2002), and U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for, e.g., antibody production, see, e.g., Yazaki and Wu, Methods in Molecular Biology, Vol. 248 (B.K.C. Lo, ed., Humana Press, Totowa, N.J.), pp. 255-268 (2003).

In addition to cloning and expression in host cells, followed by purification of the shuttle agent or portion thereof from the host cell culture, other art-known methods of producing the shuttle agents or portions thereof of the invention are known in the art. For example, proteins may be synthesized by direct peptide synthesis using solid-phase techniques (see, e.g., Stewart et al., Solid-Phase Peptide Synthesis, W.H. Freeman Co., San Francisco, Calif. (1969); Merrifield, J. Am. Chem. Soc. 85:2149-2154 [symbol] peptide synthesizer according to the manufacturer's instructions).

As described herein, the LRP8-binding molecule portion and the CNS-active compound portions of a shuttle agent may be covalently or non-covalently conjugated, and either with or without a linker. It will be understood by one of ordinary skill in the art that the foregoing methods of manufacture of shuttle agents apply equivalently to production of a component of a shuttle agent separately from other portions of the same shuttle agent. When different portions of a single shuttle agent comprised of non-covalent conjugation between the LRP8-binding molecule portion and the CNS-active compound portion are produced separately, they can be later combined into a single shuttle vector using known chemical and molecular biological techniques according to the specific conjugation method employed in the particular shuttle agent.

C. Assays

Shuttle agents provided herein may be identified, screened for, or characterized for their physical/chemical properties and/or biological activities by various assays known in the art.
1. Binding Assays and Other Assays

The LRP8-binding molecule portion of the shuttle agent binds LRP8, and that binding activity can be measured. In certain embodiments, the CNS-active portion of a shuttle agent is a binding molecule that specifically binds to one or more CNS targets. Thus, in one embodiment, a shuttle agent of the invention is tested for its target binding activity (to, i.e., LRP8 and/or a CNS target), e.g., by known methods such as ELISA, Western blot, etc.

In another aspect, competition assays may be used to identify a shuttle agent that competes with other known LRP8 or CNS target binding agents for binding to LRP8 or the CNS target. In certain embodiments, such a competing shuttle agent binds to the same epitope (e.g., a linear or a conformational epitope) that is bound by the known LRP8 or CNS target binding agents. Detailed exemplary methods for mapping an epitope to which an antibody or other binding protein binds are provided in Morris (1996) “Epitope Mapping Protocols,” in Methods in Molecular Biology vol. 66 (Humana Press, Totowa, N.J.).

In an exemplary competition assay, immobilized LRP8 or CNS target is incubated in a solution comprising a first labeled binding agent (i.e., antibody) that binds to LRP8 or the CNS target and a second unlabeled shuttle agent that is being tested for its ability to compete with the first binding agent for binding to LRP8 or the CNS target. As a control, immobilized LRP8 or CNS target is incubated in a solution comprising th [symbol] second unlabeled shuttle agent. After incubation under conditions permissive for binding of the first binding agent to LRP8 or CNS target, excess unbound binding agent is removed, and the amount of label associated with immobilized LRP8 or CNS target is measured. If the amount of label associated with immobilized LRP8 or CNS target is substantially reduced in the test sample relative to the control sample, then that indicates that the shuttle agent is competing with the first binding agent for binding to LRP8 or CNS target. See Harlow and Lane (1988) Antibodies: A Laboratory Manual ch.14 (Cold Spring Harbor Laboratory, Cold Spring Harbor, N.Y.).
2. Activity Assays

In one aspect, assays are provided for identifying shuttle agents having biological activity, i.e., wherein the CNS-active compound portion of the shuttle agent exerts an effect on one or more CNS targets. Biological activity may include, e.g., an enzymatic activity, an inhibitory (antagonistic) activity, a stimulatory (agonistic) activity, a transport activity, and a structural activity. Shuttle agents having such biological activity in vivo and/or in vitro are also provided.

In certain embodiments, a shuttle agent of the invention is tested for such biological activity in vitro. Depending on the nature of the CNS-active compound portion of the shuttle agent, a variety of art-known assays are available, and it is well-known how to select an appropriate art-known assay to test the activity of a particular CNS-active compound comprised within a shuttle agent. For example, if the shuttle agent includes a CNS-active compound portion that is an enzyme, the enzymatic activity of the shuttle agent can be readily assessed using well-known enzymatic assays (i.e., assays for kinases, phosphatases, proteases, lipases, and the like), many of which are commercially available in kit form. As another nonlimiting example, for shuttle agents comprising a CNS-active compound portion that is an inhibitor or an activator of a particular CNS target, in vitro assays measuring the ability of that shuttle agent to inhibit or stimulate the activity of that CNS target can be performed (i.e., for a peptide, aptamer or small molecule inhibitor or activator of a CNS target, the activity of the CNS target in the presence and absence of the peptide, aptamer or small molecule inhibitor or activator can be measured in vitro).

In certain embodiments, a shuttle agent of the invention is tested for such biological activity in vivo. A variety of animal models can also be used to test the efficacy of the candidate therapeutic agents. The in vivo nature of such models makes them particularly predictive of responses in human patients. For examp [symbol] neurodegenerative conditions and associated techniques for examining the pathological processes associated with these models of neurodegeneration (e.g. in the presence and absence of shuttle agent) are readily available in the art. Animal models of various neurological disorders include both non-recombinant and recombinant (transgenic) animals. Non-recombinant animal models include, for example, rodent, e.g., murine models. Such models can be generated by introducing cells into syngeneic mice using standard techniques, e.g. subcutaneous injection, tail vein injection, spleen implantation, intraperitoneal implantation, and implantation under the renal capsule. In vivo models include models of stroke/cerebral ischemia, in vivo models of neurodegenerative diseases, such as mouse models of Parkinson's disease; mouse models of Alzheimer's disease; mouse models of amyotrophic lateral sclerosis; mouse models of spinal muscular atrophy; mouse/rat models of focal and global cerebral ischemia, for instance, common carotid artery occlusion or middle cerebral artery occlusion models; or in ex vivo whole embryo cultures. As one nonlimiting example, there are a number of art-known mouse models for Alzheimer's disease ((see, e.g. Rakover et al., Neurodegener. Dis. (2007); 4(5): 392-402; Mouri et al., FASEB J. (2007) July; 21 (9): 2135-48; Minkeviciene et al ., J. Pharmacol. Exp. Ther. (2004) November; 311 (2) :677-82 and Yuede et al., Behav Pharmacol. (2007) September; 18 (5-6): 347-63). The various assays maybe conducted in known in vitro or in vivo assay formats, as known in the art and described in the literature. Various such animal models are also available from commercial vendors such as the Jackson Laboratory.

When a shuttle agent is administered to a human to treat a CNS disease or disorder characterized by cognitive impairment (i.e., Alzheimer's disease or mild cognitive impairment), one or more cognitive outcome measures in conjunction with a global assessment can be used to assess the efficacy of the shuttle agent (see, e.g., Leber P: Guidelines for the Clinical Evaluation of Antidementia Drugs, 1st draft, Rockville, Md., US Food and Drug Administration, 1990). The effects on neurological disorders, such as AD, can be examined for instance using single or multiple sets of criteria. For example, the European Medicine Evaluation Agency (EMEA) introduced a definition of responders corresponding to a prespecified degree of improvement in cognition and stabilization in both functional and global activities (see, e.g. European Medicine Evaluation Agency (EMEA): Note for Guidelines on Medicinal Products in the Treatment of Alzheimer's Disease. London, EMEA, 1997). A number of specific established tests that can be used alone or in combination to evaluate a patient's responsiveness to an agent are known in the art (see, e.g. Van Dyke et al., Am. J. Geriatr. Psychiatry 14:5 (2006). For example, responsiveness to an agent can be evaluated using the Severe Impairment Battery (SIB), a test used [symbol] with more more severe AD (see, e.g. Schmitt et al., Alzheimer Dis. Assoc. Disord. 1997; 11 (suppl 2):51-56). Responsiveness to an agent can also be measured using the 19-item Alzheimer's Disease Cooperative Study-Activities of Daily Living inventory (ADCSADL19), a 19- item inventory that measures the level of independence in performing activities of daily living, designed and validated for later stages of dementia (see, e.g. Galasko et al., J. Int. Neuropsychol. Soc. (2005); 11: 446-453). Responsiveness to an agent can also be measured using the Clinician's Interview-Based Impression of Change Plus Caregiver Input (CIBIC-Plus), a seven-point global change rating based on structured interviews with both patient and caregiver (see, e.g. Schneider et al ., Alzheimer Dis Assoc Disord 1997; 11 (suppl 2):22-32).

Responsiveness to an agent can also be measured using the Neuropsychiatric Inventory (NPI), which assesses the frequency and severity of 12 behavioral symptoms based on a caregiver interview (see, e.g. Cummings et al., Neurology 1994; 44:2308- 2314).

D. Conjugation

As described herein, a shuttle agent of the invention comprises an LRP8-binding molecule portion and a CNS-active compound portion. The two portions are conjugated into a single shuttle agent. Such conjugation may be covalent or non-covalent, and will appropriately depend on the specific LRP8-binding molecule and CNS-active compounds under consideration for conjugation.

Covalent conjugation can either be direct or via a linker. In certain embodiments, direct conjugation is by construction of a protein fusion (i.e., by genetic fusion of the two genes encoding LRP8-binding molecule and CNS-active compound and expression as a single protein). In certain embodiments, direct conjugation is by formation of a covalent bond between a reactive group on one of the two portions of the shuttle agent and a corresponding group or acceptor on the other portion of the shuttle agent. In certain embodiments, direct conjugation is by modification (i.e., genetic modification) of one of the two molecules to be conjugated to include a reactive group (as nonlimiting examples, a sulfhydryl group or a carboxyl group) that forms a covalent attachment to the other molecule to be conjugated under appropriate conditions. As one nonlimiting example, an molecule (i.e., an amino acid) with a desired reactive group (i.e., a cysteine residue) may be introduced into, i.e., the LRP8-binding molecule portion and a disulfide bond formed with the CNS-active compound portion. Methods for covalent conjugation of nucleic acids to proteins are also known in the art (i.e., photocrosslinking, see, e.g., Zatsepin et at (2005) Russ. Chem. Rev. 74: 77-95) Non-covalent conjugation can be by any nonconvalent attachment m [symbol] bonds, electrostatic interactions, and the like, as will be readily understood by one of ordinary skill in the art. Conjugation may also be performed using a variety of linkers. For example, an LRP8-binding molecule portion and a CNS-active compound portion of a shuttle agent may be conjugated using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Peptide linkers, comprised of from one to twenty amino acids joined by peptide bonds, may also be used. In certain such embodiments, the amino acids are selected from the twenty naturally-occurring amino acids. In certain other such embodiments, one or more of the amino acids are selected from glycine, alanine, proline, asparagine, glutamine and lysine. The linker may be a “cleavable linker” facilitating release of the CNS-active compound upon delivery to the brain. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) maybe used.

The shuttle agents herein expressly contemplate, but are not limited to, such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

E. Methods and Compositions for Diagnostics and Detection

In certain embodiments, labeled shuttle agents are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), lucif [symbol] horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

In certain embodiments, any of the shuttle agents provided herein is useful for detecting the presence of LRP8 and/or one or more CNS targets in a biological sample or subject. The term “detecting” as used herein encompasses quantitative or qualitative detection. In certain embodiments, a biological sample comprises a cell or tissue, such as cerebrospinal fluid, neural cells, or brain tissue.

In one embodiment, a shuttle agent for use in a method of in vitro diagnosis or detection is provided. In a further aspect, a method of detecting the presence of LRP8 in a biological sample is provided. In a further aspect, a method of detecting the presence of one or more CNS targets in a sample is provided. In certain embodiments, the method comprises contacting the biological sample with a shuttle agent as described herein under conditions permissive for binding of the shuttle agent to LRP8 and/or one or more CNS targets, and detecting whether a complex is formed between the shuttle agent and LRP8 and/or one or more CNS targets. Such method may be an in vitro or in vivo method. In one embodiment, shuttle agent is used to select subjects eligible for therapy with a shuttle agent, e.g. where a CNS target is a biomarker for selection of patients.

In one embodiment, a shuttle agent for use in a method of in vivo diagnosis or detection is provided. In one aspect, a method of detecting the presence, amount and/or activity of one or more CNS targets in a subject is provided. In certain aspects, a shuttle agent is administered to a subject and the localization of the shuttle agent within the CNS is detected. In certain such aspects, the shuttle agent is labeled. In certain such aspects, the detection is by noninvasive imaging means (i.e., radiography, tomography, magnetic resonance imaging, or other imaging technique). In certain aspects, the detection is by assessing one or more biological samples from the subject to which the shuttle agent had been administered for shuttle agent presence and/or activity. In certain aspects, the amount of the shuttle agent within the CNS is measured in addition to or instead of the localization of the shuttle agent within the CNS. In certain aspects, the activity of the shuttle agent within the CNS is measured (i.e., as will be understood by one of ordinary skill in the art, the label on the shuttle agent may be selected to be detectable only if the CNS-active portion of the shuttle agent is a [symbol] human. In certain aspects, the detection is for routine assessment of the status of the CNS in the subject. In certain aspects, the detection is for detection of degeneration or injury within the CNS. In certain aspects, the detection is for early detection of one or more indicators of the presence of a CNS disease or disorder. In certain aspects, the detection is to assess the severity and/or progress of a CNS disease or disorder. In certain aspects, the detection is to assess the efficacy or impact of a particular therapy or therapies. It will be appreciated by one of ordinary skill in the art that any of the foregoing types of detections may benefit from or involve a series of detections collected over a given time frame, or comparison of the results of a particular detection to those from healthy subjects or control subjects with known levels of a CNS disease or disorder.

Exemplary diseases and disorders that may be diagnosed using an antibody of the invention include neuropathy, amyloidosis, cancer, viral or microbial infection, inflammation, ischemia, neurodegenerative disease, seizure, behavioral disorders, and a lysosomal storage disease.

F. Pharmaceutical Formulations

Pharmaceutical formulations of a shuttle agent (i.e., an anti-LRP8 antibody conjugated to a CNS-active compound) as described herein are prepared by mixing such shuttle agent having the desired degree of purity with one or more optional pharmaceutically acceptable carriers (Remington's Pharmaceutical Sciences 20th edition, Osol, A. Ed.: Williams and Wilkins Pa., USA (2000)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids and their salts; mineral acid salts such as hydrochlorides; hydrobromides; and sulfates; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; thimerosal; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such a [symbol] protein complexes); agents delaying absorption such as aluminum monostearate; and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. For example, it may be desirable to further provide one or more compounds appropriate to treat the same or a different CNS disease or disorder as the shuttle agent is intended to treat. Similarly, it may be desirable to further provide one or more compounds appropriate to diagnose the same or a different CNS disease or disorder as the shuttle agent is intended to diagnose. Appropriate additional compounds will be well-known to those of ordinary skill in the art. For example, when a shuttle agent is a bispecific antibody targeting LRP8 and a CNS target intended to treat Alzheimer's disease, such as amyloid precursor protein, amyloid beta peptide (monomeric, oligomeric, or fibril forms), beta secretase, gamma secretase, and the like, the formulation for that shuttle agent may also comprise one or more additional Alzheimer's therapeutic (i.e., a cholinesterase inhibitor, memantine, an anti-agitation medication, an anti-depressive, an anxiolytic, and the like). Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepare [symbol] preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

G. Therapeutic Methods and Compositions

The shuttle agents of the invention may be used as therapeutic compositions. In certain embodiments, a shuttle agent of the invention is used to treat or prevent one or more CNS diseases or disorders. Exemplary CNS diseases or disorders include, but are not limited to, neuropathy, amyloidosis, cancer, viral or microbial infection, inflammation, ischemia, neurodegenerative disease, seizure, behavioral disorders, and a lysosomal storage disease.

Neuropathy disorders are diseases or abnormalities of the nervous system characterized by inappropriate or uncontrolled nerve signaling or lack thereof, and include, but are not limited to, chronic pain (including nociceptive pain (pain caused by an injury to body tissues, including cancer-related pain), neuropathic pain (pain caused by abnormalities in the nerves, spinal cord, or brain), and psychogenic pain (entirely or mostly related to a psychological disorder), headache, migraine, neuropathy, and symptoms and syndromes often accompanying such neuropathy disorders such as vertigo or nausea.

Amyloidoses are a group of diseases and disorders associated with extracellular proteinaceous deposits in the CNS, including, but not limited to, secondary amyloidosis, age-related amyloidosis, Alzheimer's Disease (AD), mild cognitive impairment (MCI), Lewy body dementia, Down's syndrome, hereditary cerebral hemorrhage with amyloidosis (Dutch type); the Guam Parkinson-Dementia complex, cerebral amyloid angiopathy, Huntington's disease, progressive supranuclear palsy, multiple sclerosis; Creutzfeld Jacob disease, Parkinson's disease, transmissible spongiform encephalopathy, HIV-related dementia, amyotropic lateral sclerosis (ALS), inclusion-body myositis (IBM), and ocular diseases relating to beta-amyloid deposition (i.e., macular degeneration, drusen-related optic neuropathy, and cataract).

Cancers of the CNS are characterized by aberrant proliferation of one or more CNS cell (i.e., a neural cell) and include, but are not limited to, glioma, glioblastoma multiforme, meningioma, astrocytoma, acoustic neuroma, chondroma, oligodendroglioma, medulloblastomas, ganglioglioma, Schwannoma, neurofibroma, neuroblastoma, and extradural, intramedullary or intradural tumors.

Ocular diseases or disorders are diseases or dis [symbol] herein is considered a CNS organ subject to the BBB. Ocular diseases or disorders include, but are not limited to, disorders of sclera, cornea, iris and ciliary body (i.e., scleritis, keratitis, corneal ulcer, corneal abrasion, snow blindness, arc eye, Thygeson's superficial punctate keratopathy, corneal neovascularisation, Fuchs' dystrophy, keratoconus, keratoconjunctivitis sicca, iritis and uveitis), disorders of the lens (i.e., cataract), disorders of choroid and retina (i.e., retinal detachment, retinoschisis, hypertensive retinopathy, diabetic retinopathy, retinopathy, retinopathy of prematurity, age-related macular degeneration, macular degeneration (wet or dry), epiretinal membrane, retinitis pigmentosa and macular edema), glaucoma, floaters, disorders of optic nerve and visual pathways (i.e., Leber's hereditary optic neuropathy and optic disc drusen), disorders of ocular muscles/binocular movement accommodation/refraction (i.e., strabismus, ophthalmoparesis, progressive external opthalmoplegia, esotropia, exotropia, hypermetropia, myopia, astigmatism, anisometropia, presbyopia and ophthalmoplegia), visual disturbances and blindness (i.e., amblyopia, Lever's congenital amaurosis, scotoma, color blindness, achromatopsia, nyctalopia, blindness, river blindness and micro-opthalmia/coloboma), red eye, Argyll Robertson pupil, keratomycosis, xerophthalmia and andaniridia.

Viral or microbial infections of the CNS include, but are not limited to, infections by viruses (i.e., influenza, HIV, poliovirus, rubella,), bacteria (i.e., Neisseria sp., Streptococcus sp., Pseudomonas sp., Proteus sp., E. coli, S. aureus, Pneumococcus sp., Meningococcus sp., Haemophilus sp., and Mycobacterium tuberculosis) and other microorganisms such as fungi (i.e., yeast, Cryptococcus neoformans), parasites (i.e., toxoplasma gondii) or amoebas resulting in CNS pathophysiologies including, but not limited to, meningitis, encephalitis, myelitis, vasculitis and abscess, which can be acute or chronic.

Inflammation of the CNS is inflammation that is caused by an injury to the CNS, which can be a physical injury (i.e., due to accident, surgery, brain trauma, spinal cord injury, concussion) or an injury due to or related to one or more other diseases or disorders of the CNS (i.e., abscess, cancer, viral or microbial infection).

Ischemia of the CNS, as used herein, refers to a group of disorders relating to aberrant blood flow or vascular behavior in the brain or the causes therefor, and includes, but is not limited to, focal brain ischemia, global brain ischemia, stroke (i.e., subarachnoid hemorrhage and intracerebral hemorrhage), and aneurysm.

Neurodegenerative diseases are a group of diseases and disorders associated with neural cell loss of function or death in the CNS, and include, but are not limited to, adrenoleukodystrophy, Alexander's disease, Alper's d [symbol] ataxia telangiectasia, Batten disease, cockayne syndrome, corticobasal degeneration, degeneration caused by or associated with an amyloidosis, Friedreich's ataxia, frontotemporal lobar degeneration, Kennedy's disease, multiple system atrophy, multiple sclerosis, primary lateral sclerosis, progressive supranuclear palsy, spinal muscular atrophy, transverse myelitis, Refsum's disease, and spinocerebellar ataxia.

Seizure diseases and disorders of the CNS involve inappropriate and/or abnormal electrical conduction in the CNS, and include, but are not limited to, epilepsy (i.e., absence seizures, atonic seizures, benign Rolandic epilepsy, childhood absence, clonic seizures, complex partial seizures, frontal lobe epilepsy, febrile seizures, infantile spasms, juvenile myoclonic epilepsy, juvenile absence epilepsy, Lennox-Gastaut syndrome, Landau-Kleffner Syndrome, Dravet's syndrome, Otahara syndrome, West syndrome, myoclonic seizures, mitochondrial disorders, progressive myoclonic epilepsies, psychogenic seizures, reflex epilepsy, Rasmussen's Syndrome, simple partial seizures, secondarily generalized seizures, temporal lobe epilepsy, toniclonic seizures, tonic seizures, psychomotor seizures, limbic epilepsy, partial-onset seizures, generalized-onset seizures, status epilepticus, abdominal epilepsy, akinetic seizures, autonomic seizures, massive bilateral myoclonus, catamenial epilepsy, drop seizures, emotional seizures, focal seizures, gelastic seizures, Jacksonian March, Lafora Disease, motor seizures, multifocal seizures, nocturnal seizures, photosensitive seizure, pseudo seizures, sensory seizures, subtle seizures, sylvan seizures, withdrawal seizures, and visual reflex seizures)

Behavioral disorders are disorders of the CNS characterized by aberrant behavior on the part of the afflicted subject and include, but are not limited to, sleep disorders (i.e., insomnia, parasomnias, night terrors, circadian rhythm sleep disorders, and narcolepsy), mood disorders (i.e., depression, suicidal depression, anxiety, chronic affective disorders, phobias, panic attacks, obsessive-compulsive disorder, attention deficit hyperactivity disorder (ADHD), attention deficit disorder (ADD), chronic fatigue syndrome, agoraphobia, post-traumatic stress disorder, bipolar disorder), eating disorders (i.e., anorexia or bulimia), psychoses, developmental behavioral disorders (i.e., autism, Rett's syndrome, Aspberger's syndrome), personality disorders and psychotic disorders (i.e., schizophrenia, delusional disorder, and the like).

Lysosomal storage disorders are metabolic disorders which are in some cases associated with the CNS or have CNS-specific symptoms; such disorders include, but are not limited to Tay-Sachs disease, Gaucher's disease, Fabry disease, mucopolysaccharidosis (types I, II, III, IV, V, VI and VII), glycogen storage disease, GM1-ga [symbol] leukodystrophy, Farber's disease, Canavan's leukodystrophy, and neuronal ceroid lipofuscinoses types 1 and 2, Niemann-Pick disease, Pompe disease, and Krabbe's disease.

Any of the shuttle agents provided herein may be used in therapeutic methods. In one aspect, a shuttle agent for use as a medicament is provided. In further aspects, a shuttle agent for use in treating a CNS disease or disorder is provided. In certain embodiments, a shuttle agent for use in a method of treatment is provided. In certain embodiments, the invention provides a shuttle agent for use in a method of treating an individual having a CNS disease or disorder comprising administering to the individual an effective amount of the shuttle agent. In one such embodiment the CNS disease or disorder is selected from a neuropathy, amyloidosis, cancer, viral or microbial infection, inflammation, ischemia, neurodegenerative disease, seizure, behavioral disorders, and a lysosomal storage disease, as further explicated above. In one such embodiment, the method further comprises administering to the individual an effective amount of at least one additional therapeutic agent, e.g., as described below. An “individual” according to any of the above embodiments is preferably a human.

In a further aspect, the invention provides for the use of a shuttle agent in the manufacture or preparation of a medicament. In one embodiment, the medicament is for treatment of a CNS disease or disorder. In a further embodiment, the medicament is for use in a method of treating a CNS disease or disorder comprising administering to an individual having a CNS disease or disorder an effective amount of the medicament. In one such embodiment, the method further comprises administering to the individual an effective amount of at least one additional therapeutic agent, e.g., as described below. In any of such embodiments, the CNS disease or disorder is selected from a neuropathy, amyloidosis, cancer, viral or microbial infection, inflammation, ischemia, neurodegenerative disease, seizure, behavioral disorders, and a lysosomal storage disease. An “individual” according to any of the above embodiments may be a human.

In a further aspect, the invention provides a method for treating a CNS disease or disorder. In one embodiment, the method comprises administering to an individual having such CNS disease or disorder an effective amount of a shuttle agent. In one such embodiment, the method further comprises administering to the individual an effective amount of at least one additional therapeutic agent, as described below. In any of such embodiments, the CNS disease or disorder is selected from a neuropathy, amyloidosis, cancer, viral or microbial infection, inflammation, ischemia, neurodegenerative disease, seizure, behavioral disorders, and a lysosomal storage disease. An “individual” acc [symbol] may be a human.

In a further aspect, the invention provides pharmaceutical formulations comprising any of the shuttle agents provided herein, e.g., for use in any of the above therapeutic methods. In one embodiment, a pharmaceutical formulation comprises any of the shuttle agents provided herein and a pharmaceutically acceptable carrier. In another embodiment, a pharmaceutical formulation comprises any of the shuttle agents provided herein and at least one additional therapeutic agent, e.g., as described below.

The CNS-active compound portion of a particular shuttle agent can be selected from compounds known to those of ordinary skill in the art to be useful for the detection, prevention and/or treatment of a particular CNS disease or disorder. A given CNS-active compound may have efficacy or utility in detecting, preventing and/or treating more than one CNS disease or disorder.

For a neuropathy disorder, a CNS-active compound may be selected that is an analgesic including, but not limited to, a narcotic/opioid analgesic (i.e., morphine, fentanyl, hydrocodone, meperidine, methadone, oxymorphone, pentazocine, propoxyphene, tramadol, codeine and oxycodone), a nonsteroidal anti-inflammatory drug (NSAID) (i.e., ibuprofen, naproxen, diclofenac, diflunisal, etodolac, fenoprofen, flurbiprofen, indomethacin, ketorolac, mefenamic acid, meloxicam, nabumetone, oxaprozin, piroxicam, sulindac, and tolmetin), a corticosteroid (i.e., cortisone, prednisone, prednisolone, dexamethasone, methylprednisolone and triamcinolone), an anti-migraine agent (i.e., sumatriptin, almotriptan, frovatriptan, sumatriptan, rizatriptan, eletriptan, zolmitriptan, dihydroergotamine, eletriptan and ergotamine), acetaminophen, a salicylate (i.e., aspirin, choline salicylate, magnesium salicylate, diflunisal, and salsalate), a anti-convulsant (i.e., carbamazepine, clonazepam, gabapentin, lamotrigine, pregabalin, tiagabine, and topiramate), an anaesthetic (i.e., isoflurane, trichloroethylene, halothane, sevoflurane, benzocaine, chloroprocaine, cocaine, cyclomethycaine, dimethocaine, propoxycaine, procaine, novocaine, proparacaine, tetracaine, articaine, bupivacaine, carticaine, cinchocaine, etidocaine, levobupivacaine, lidocaine, mepivacaine, piperocaine, prilocaine, ropivacaine, trimecaine, saxitoxin and tetrodotoxin), and a cox-2-inhibitor (i.e., celecoxib, rofecoxib, and valdecoxib). For a neuropathy disorder with vertigo involvement, a CNS-active compound may be selected that is an anti-vertigo agent including, but not limited to, meclizine, diphenhydramine, promethazine and diazepam. For a neuropathy disorder with nausea involvement, a CNS-active compound may be selected that is an anti-nausea agent including, but not limited to, promethazine, chlorpromazine, prochlorperazine, trimethobenzamide, and metoclopramide. For a neurodegenerative disease, a [symbol] that is a growth hormone or neurotrophic factor; examples include but are not limited to brain-derived neurotrophic factor (BDNF), nerve growth factor (NGF), neurotrophin-4/5, fibroblast growth factor (FGF)-2 and other FGFs, neurotrophin (NT)-3, erythropoietin (EPO), hepatocyte growth factor (HGF), epidermal growth factor (EGF), transforming growth factor (TGF)-alpha, TGF-beta, vascular endothelial growth factor (VEGF), interleukin-1 receptor antagonist (IL-1ra), ciliary neurotrophic factor (CNTF), glial-derived neurotrophic factor (GDNF), neurturin, platelet-derived growth factor (PDGF), heregulin, neuregulin, artemin, persephin, interleukins, glial cell line derived neurotrophic factor (GFR), granulocyte-colony stimulating factor (CSF), granulocyte-macrophage-CSF, netrins, cardiotrophin-1, hedgehogs, leukemia inhibitory factor (LIF), midkine, pleiotrophin, bone morphogenetic proteins (BMPs), netrins, saposins, semaphorins, and stem cell factor (SCF).

For cancer, a CNS-active compound may be selected that is a chemotherapeutic agent. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphor-amide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gammall and calicheamicin omegall (see, e.g., Agnew, Chem Intl. Ed. Engl., 33: 183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, ca [symbol] chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; gemcitabine (GEMZAR®); 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorometlhylornithine (DMFO); retinoids such as retinoic acid; capecitabine (XELODA®); pharmaceutically acceptable salts, acids or derivatives of any of the above; as well as combinations of two or more of the above such as CHOP, an abbreviation for a combined therapy of cyclophosphamide, doxorubicin, vincristin [symbol] abbreviation for a treatment regimen with oxaliplatin (ELOXATIN™) combined with 5-FU and leucovovin.

Also included in this definition of chemotherapeutic agents are anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), EVISTA® raloxifene, droloxifene, 4-hydroxytamoxifen, trioxifene, keoxifene, LY117018, onapristone, and FARESTON® toremifene; anti-progesterones; estrogen receptor down-regulators (ERDs); agents that function to suppress or shut down the ovaries, for example, leutinizing hormone-releasing hormone (LHRH) agonists such as LUPRON® and ELIGARD® leuprolide acetate, goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole. In addition, such definition of chemotherapeutic agents includes bisphosphonates such as clodronate (for example, BONEFOS® or OSTAC®), DIDROCAL® etidronate, NE-58095, ZOMETA® zoledronic acid/zoledronate, FOSAMAX® alendronate, AREDIA® pamidronate, SKELID® tiludronate, or ACTONEL® risedronate; as well as troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; LURTOTECAN® topoisomerase 1 inhibitor; ABARELIX® rmRH; lapatinib ditosylate (an ErbB-2 and EGFR dual tyrosine kinase small-molecule inhibitor also known as GW572016); and pharmaceutically acceptable salts, acids or derivatives of any of the above.

Another group of compounds that may be selected as CNS-active compounds for cancer treatment or prevention are anti-cancer immunoglobulins (including, but not limited to, trastuzumab, bevacizumab, alemtuxumab, cetuximab, gemtuzumab ozogamicin, ibritumomab tiuxetan, panitumumab and rituximab). In some instances, antibodies in conjunction with a toxic label may be used to target and kill desired cells [symbol] limited to, tositumomab with an ¹³¹I radiolabel.

For an ocular disease or disorder, a CNS-active compound may be selected that is an anti-angiogenic ophthalmic agent (i.e., bevacizumab, ranibizumab and pegaptanib), an ophthalmic glaucoma agent (i.e., carbachol, epinephrine, demecarium bromide, apraclonidine, brimonidine, brinzolamide, levobunolol, timolol, betaxolol, dorzolamide, bimatoprost, carteolol, metipranolol, dipivefrin, travoprost and latanoprost), a carbonic anhydrase inhibitor (i.e., methazolamide and acetazolamide), an ophthalmic antihistamine (i.e., naphazoline, phenylephrine and tetrahydrozoline), an ocular lubricant, an ophthalmic steroid (i.e., fluorometholone, prednisolone, loteprednol, dexamethasone, difluprednate, rimexolone, fluocinolone, medrysone and triamcinolone), an ophthalmic anesthetic (i.e., lidocaine, proparacaine and tetracaine), an ophthalmic anti-infective (i.e., levofloxacin, gatifloxacin, ciprofloxacin, moxifloxacin, chloramphenicol, bacitracin/polymyxin b, sulfacetamide, tobramycin, azithromycin, besifloxacin, norfloxacin, sulfisoxazole, gentamicin, idoxuridine, erythromycin, natamycin, gramicidin, neomycin, ofloxacin, trifluridine, ganciclovir, vidarabine), an ophthalmic anti-inflammatory agent (i.e., nepafenac, ketorolac, flurbiprofen, suprofen, cyclosporine, triamcinolone, diclofenac and bromfenac), and an ophthalmic antihistamine or decongestant (i.e., ketotifen, olopatadine, epinastine, naphazoline, cromolyn, tetrahydrozoline, pemirolast, bepotastine, naphazoline, phenylephrine, nedocromil, lodoxamide, phenylephrine, emedastine and azelastine).

For a seizure disorder, a CNS-active compound may be selected that is an anticonvulsant or antiepileptic including, but not limited to, barbiturate anticonvulsants (i.e., primidone, metharbital, mephobarbital, allobarbital, amobarbital, aprobarbital, alphenal, barbital, brallobarbital and phenobarbital), benzodiazepine anticonvulsants (i.e., diazepam, clonazepam, and lorazepam), carbamate anticonvulsants (i.e. felbamate), carbonic anhydrase inhibitor anticonvulsants (i.e., acetazolamide, topiramate and zonisamide), dibenzazepine anticonvulsants (i.e., rufinamide, carbamazepine, and oxcarbazepine), fatty acid derivative anticonvulsants (i.e., divalproex and valproic acid), gamma-aminobutyric acid analogs (i.e., pregabalin, gabapentin and vigabatrin), gamma-aminobutyric acid reuptake inhibitors (i.e., tiagabine), gamma-aminobutyric acid transaminase inhibitors (i.e., vigabatrin), hydantoin anticonvulsants (i.e. phenytoin, ethotoin, fosphenytoin and mephenytoin), miscellaneous anticonvulsants (i.e., lacosamide and magnesium sulfate), progestins (i.e., progesterone), oxazolidinedione anticonvulsants (i.e., paramethadione and trimethadione), pyrrolidine anticonvulsants (i.e., levetiracetam), succinimide anticonvulsants (i.e., ethosuximide and methsuximide), triazine anticonvulsants (i.e., lamotrig [symbol] phenacemide and pheneturide).

For a lysosomal storage disease, a CNS-active compound may be selected that is itself or otherwise mimics the activity of the enzyme that is impaired in the disease. Exemplary recombinant enzymes for the treatment of lysosomal storage disorders include, but are not limited to those set forth in e.g., U.S. Patent Application publication no. 20050142141 (i.e., alpha-L-iduronidase, iduronate-2-sulphatase, N-sulfatase, alpha-N-acetylglucosaminidase, N-acetyl-galactosamine-6-sulfatase, beta-galactosidase, arylsulphatase B, beta-glucuronidase, acid alpha-glucosidase, glucocerebrosidase, alpha-galactosidase A, hexosaminidase A, acid sphingomyelinase, beta-galactocerebrosidase, beta-galactosidase, arylsulfatase A, acid ceramidase, aspartoacylase, palmitoyl-protein thioesterase 1 and tripeptidyl amino peptidase 1).

For amyloidosis, a CNS-active compound may be selected that includes, but is not limited to, an antibody or other binding molecule (including, but not limited to a small molecule, a peptide, an aptamer, or other protein binder) that specifically binds to a target selected from: beta secretase, tau, presenilin, amyloid precursor protein or portions thereof, amyloid beta peptide or oligomers or fibrils thereof, death receptor 6 (DR6), receptor for advanced glycation endproducts (RAGE), parkin, and huntingtin; a cholinesterase inhibitor (i.e., galantamine, donepezil, rivastigmine and tacrine); an NMDA receptor antagonist (i.e., memantine), a monoamine depletor (i.e., tetrabenazine); an ergoloid mesylate; an anticholinergic antiparkinsonism agent (i.e., procyclidine, diphenhydramine, trihexylphenidyl, benztropine, biperiden and trihexyphenidyl); a dopaminergic antiparkinsonism agent (i.e., entacapone, selegiline, pramipexole, bromocriptine, rotigotine, selegiline, ropinirole, rasagiline, apomorphine, carbidopa, levodopa, pergolide, tolcapone and amantadine); a tetrabenazine; an anti-inflammatory (including, but not limited to, a nonsteroidal anti-inflammatory drug (i.e., indomethicin and other compounds listed above); a hormone (i.e., estrogen, progesterone and leuprolide); a vitamin (i.e., folate and nicotinamide); a dimebolin; a homotaurine (i.e., 3-aminopropanesulfonic acid; 3APS); a serotonin receptor activity modulator (i.e., xaliproden); an, an interferon, and a glucocorticoid.

For a viral or microbial disease, a CNS-active compound may be selected that includes, but is not limited to, an antiviral compound (including, but not limited to, an adamantane antiviral (i.e., rimantadine and amantadine), an antiviral interferon (i.e., peginterferon alfa-2b), a chemokine receptor antagonist (i.e., maraviroc), an integrase strand transfer inhibitor (i.e., raltegravir), a neuraminidase inhibitor (i.e., oseltamivir and zanamivir), a non-nucleoside reverse transcriptase inhibitor (i.e., efavirenz, etravirine, delavirdine and nevirapine), a nucleoside reverse transcriptase inhibitors (tenofovir, [symbol] stavudine, entecavir, emtricitabine, adefovir, zalcitabine, telbivudine and didanosine), a protease inhibitor (i.e., darunavir, atazanavir, fosamprenavir, tipranavir, ritonavir, nelfinavir, amprenavir, indinavir and saquinavir), a purine nucleoside (i.e., valacyclovir, famciclovir, acyclovir, ribavirin, ganciclovir, valganciclovir and cidofovir), and a miscellaneous antiviral (i.e., enfuvirtide, foscarnet, palivizumab and fomivirsen)), an antibiotic (including, but not limited to, an aminopenicillin (i.e., amoxicillin, ampicillin, oxacillin, nafcillin, cloxacillin, dicloxacillin, flucoxacillin, temocillin, azlocillin, carbenicillin, ticarcillin, mezlocillin, piperacillin and bacampicillin), a cephalosporin (i.e., cefazolin, cephalexin, cephalothin, cefamandole, ceftriaxone, cefotaxime, cefpodoxime, ceftazidime, cefadroxil, cephradine, loracarbef, cefotetan, cefuroxime, cefprozil, cefaclor, and cefoxitin), a carbapenem/penem (i.e., imipenem, meropenem, ertapenem, faropenem and doripenem), a monobactam (i.e., aztreonam, tigemonam, norcardicin A and tabtoxinine-beta-lactam, a beta-lactamase inhibitor (i.e., clavulanic acid, tazobactam and sulbactam) in conjunction with another beta-lactam antibiotic, an aminoglycoside (i.e., amikacin, gentamicin, kanamycin, neomycin, netilmicin, streptomycin, tobramycin, and paromomycin), an ansamycin (i.e., geldanamycin and herbimycin), a carbacephem (i.e., loracarbef), a glycopeptides (i.e., teicoplanin and vancomycin), a macrolide (i.e., azithromycin, clarithromycin, dirithromycin, erythromycin, roxithromycin, troleandomycin, telithromycin and spectinomycin), a monobactam (i.e., aztreonam), a quinolone (i.e., ciprofloxacin, enoxacin, gatifloxacin, levofloxacin, lomefloxacin, moxifloxacin, norfloxacin, ofloxacin, trovafloxacin, grepafloxacin, sparfloxacin and temafloxacin), a sulfonamide (i.e., mafenide, sulfonamidochrysoidine, sulfacetamide, sulfadiazine, sulfamethizole, sulfanilamide, sulfasalazine, sulfisoxazole, trimethoprim, trimethoprim and sulfamethoxazole), a tetracycline (i.e., tetracycline, demeclocycline, doxycycline, minocycline and oxytetracycline), an antineoplastic or cytotoxic antibiotic (i.e., doxorubicin, mitoxantrone, bleomycin, daunorubicin, dactinomycin, epirubicin, idarubicin, plicamycin, mitomycin, pentostatin and valrubicin) and a miscellaneous antibacterial compound (i.e., bacitracin, colistin and polymyxin B)), an antifungal (i.e., metronidazole, nitazoxanide, tinidazole, chloroquine, iodoquinol and paromomycin), and an antiparasitic (including, but not limited to, quinine, chloroquine, amodiaquine, pyrimethamine, sulphadoxine, proguanil, mefloquine, atovaquone, primaquine, artemesinin, halofantrine, doxycycline, clindamycin, mebendazole, pyrantel pamoate, thiabendazole, diethylcarbamazine, ivermectin, rifampin, amphotericin B, melarsoprol, efornithine and albendazole).

For ischemia, a CNS-active compound may be [symbol] to, a thrombolytic (i.e., urokinase, alteplase, reteplase and tenecteplase), a platelet aggregation inhibitor (i.e., aspirin, cilostazol, clopidogrel, prasugrel and dipyridamole), a statin (i.e., lovastatin, pravastatin, fluvastatin, rosuvastatin, atorvastatin, simvastatin, cerivastatin and pitavastatin), and a compound to improve blood flow or vascular flexibility, including, e.g., blood pressure medications.

For a behavioral disorder, a CNS-active compound may be selected from a behavior-modifying compound including, but not limited to, an atypical antipsychotic (i.e., risperidone, olanzapine, apripiprazole, quetiapine, paliperidone, asenapine, clozapine, iloperidone and ziprasidone), a phenothiazine antipsychotic (i.e., prochlorperazine, chlorpromazine, fluphenazine, perphenazine, trifluoperazine, thioridazine and mesoridazine), a thioxanthene (i.e., thiothixene), a miscellaneous antipsychotic (i.e., pimozide, lithium, molindone, haloperidol and loxapine), a selective serotonin reuptake inhibitor (i.e., citalopram, escitalopram, paroxetine, fluoxetine and sertraline), a serotonin-norepinephrine reuptake inhibitor (i.e., duloxetine, venlafaxine, desvenlafaxine, a tricyclic antidepressant (i.e., doxepin, clomipramine, amoxapine, nortriptyline, amitriptyline, trimipramine, imipramine, protriptyline and desipramine), a tetracyclic antidepressant (i.e., mirtazapine and maprotiline), a phenylpiperazine antidepressant (i.e., trazodone and nefazodone), a monoamine oxidase inhibitor (i.e., isocarboxazid, phenelzine, selegiline and tranylcypromine), a benzodiazepine (i.e., alprazolam, estazolam, flurazeptam, clonazepam, lorazepam and diazepam), a norepinephrine-dopamine reuptake inhibitor (i.e., bupropion), a CNS stimulant (i.e., phentermine, diethylpropion, methamphetamine, dextroamphetamine, amphetamine, methylphenidate, dexmethylphenidate, lisdexamfetamine, modafinil, pemoline, phendimetrazine, benzphetamine, phendimetrazine, armodafinil, diethylpropion, caffeine, atomoxetine, doxapram, and mazindol), an anxiolytic/sedative/hypnotic (including, but not limited to, a barbiturate (i.e., secobarbital, phenobarbital and mephobarbital), a benzodiazepine (as described above), and a miscellaneous anxiolytic/sedative/hypnotic (i.e. diphenhydramine, sodium oxybate, zaleplon, hydroxyzine, chloral hydrate, aolpidem, buspirone, doxepin, eszopiclone, ramelteon, meprobamate and ethclorvynol)), a secretin (see, e.g., Ratliff-Schaub et al. (2005) Autism 9: 256-265), an opioid peptide (see, e.g., Cowen et al., (2004) J. Neurochem. 89:273-285), and a neuropeptide (see, e.g., Hethwa et al. (2005) Am. J. Physiol. 289: E301-305).

For CNS inflammation, a CNS-active compound may be selected that addresses the inflammation itself (i.e., a nonsteroidal anti-inflammatory agent such as ibuprofen or naproxen), or one which treats the underlying cause of [symbol] anti-cancer agent).

Shuttle agents of the invention can be used either alone or in combination with other agents in a therapy. For instance, a shuttle agent of the invention may be co-administered with at least one additional therapeutic agent. In certain embodiments, an additional therapeutic agent is a therapeutic agent effective to treat the same or a different CNS disease or disorder as the shuttle agent is being employed to treat. Exemplary additional therapeutic agents include, but are not limited to, the various CNS-active compounds described above, cholinesterase inhibitors (such as donepezil, galantamine, rovastigmine, and tacrine), NMDA receptor antagonists (such as memantine), amyloid beta peptide aggregation inhibitors, antioxidants, γ-secretase modulators, nerve growth factor (NGF) mimics or NGF gene therapy, PPARγ agonists, HMS-CoA reductase inhibitors (statins), ampakines, calcium channel blockers, GABA receptor antagonists, glycogen synthase kinase inhibitors, intravenous immunoglobulin, muscarinic receptor agonists, nicrotinic receptor modulators, active or passive amyloid beta peptide immunization, phosphodiesterase inhibitors, serotonin receptor antagonists and anti-amyloid beta peptide antibodies. In certain embodiments, the at least one additional therapeutic agent is selected for its ability to mitigate one or more side effects of the CNS-active compound.

Such combination therapies noted above encompass combined administration (where two or more therapeutic agents are included in the same or separate formulations), and separate administration, in which case, administration of the antibody of the invention can occur prior to, simultaneously, and/or following, administration of the additional therapeutic agent and/or adjuvant. Antibodies of the invention can also be used in combination with other interventional therapies such as, but not limited to, radiation therapy, behavioral therapy, or other therapies known in the art and appropriate for the CNS disease or disorder to be treated or prevented.

A shuttle agent of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Shuttle agents of the invention would be form [symbol] fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The shuttle agent need not be, but is optionally formulated with one or more agents currently used to prevent or treat the disorder in question. The effective amount of such other agents depends on the amount of shuttle agent present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of a shuttle agent of the invention (when used alone or in combination with one or more other additional therapeutic agents) will depend on the type of disease to be treated, the type of shuttle agent, the severity and course of the disease, whether the shuttle agent is administered for preventive or therapeutic purposes, previous therapy, the patient's clinical history and response to the shuttle agent, and the discretion of the attending physician. The shuttle agent is suitably administered to the patient at one time or over a series of treatments. Depending on the type and severity of the disease, about 1 μg/kg to 15 mg/kg (e.g. 0.1 mg/kg-10 mg/kg) of shuttle agent can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the shuttle agent would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the shuttle agent). An initial higher loading dose, followed by one or more lower doses may be administered. However, other dosage regimens may be useful. The progress of this therapy is easily monitored by conventional techniques and assays.

H. Articles of Manufacture

In another aspect of the invention, an article of manufacture containing materials useful for the treatment, prevention and/or diagnosis of the CNS diseases and disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a shuttle agent of the invention. The label or package insert indicates that the composition is used for treating the condition of choice. Moreover, the article of manufacture may comprise (a) a first container with a composition contained therein, wherein the composition comprises a shuttle agent of the invention; and (b) a second container with a composition contained therein, wherein the composition comprises a further therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### III. EXAMPLES

The following are examples of methods and compositions of the invention. It is understood that various other embodiments may be practiced, given the general description provided above.

### Example 1

### Identification of LRP8 as a Candidate BBB-Specific Translocation-Facilitating Target

Microarray gene expression studies were performed to identify proteins that were (a) specific to the blood-brain barrier, (b) demonstrated high expression levels and (c) consistently present at different points during the lifecycle of the animal. Accordingly, protein expression was analyzed in purified brain endothelial cells versus liver and lung cells of embryonic, young, and adult mice. Specifically, C57b16 adult, pu [symbol] 14.5) mice were sacrificed by standard techniques. For adults and pups, the cerebral cortex, liver and lung were harvested in ice-cold PBS. Single cell suspensions were prepared for each tissue as follows. For brain, the meninges were removed (by Whatman paper in adults and pups, and by forceps in embryos) and the cerebral cortex was dissected away from the forebrain. A papain-based neural dissociation kit (Miltenyi) was used according to the manufacturer's directions with the exception that PBS was used instead of HBSS, and cells were filtered through a 70 micron filter centrifuged at 1,200 rpm for three minutes. The kit's normal myelin-removal step was not employed in the case of the embryo samples. For lung (whole lung lobes in each of adult, pup and embryo) and liver (peripheral regions of each lobe selected to avoid the hepatic portal vein), a modified neural dissociation kit (Miltenyi) was employed in which the initial papain incubation was performed for one hour (adult), 30 minutes (pup) or 1-5 minutes (embryo) instead of the manufacturer-instructed 15 minutes. For adult samples only, the liver/lung samples were rotor homogenized prior to trituration.

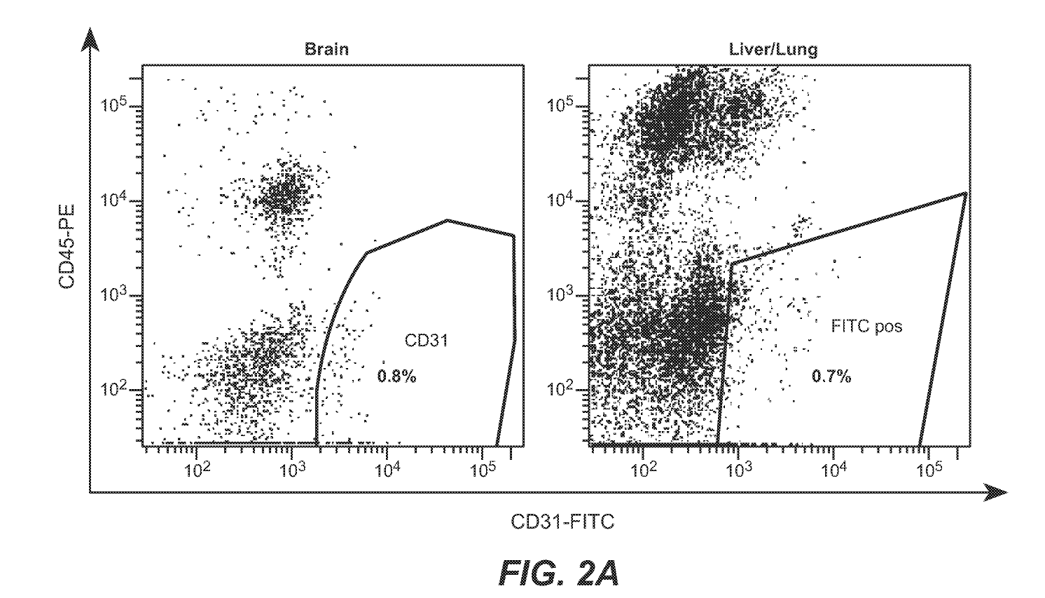

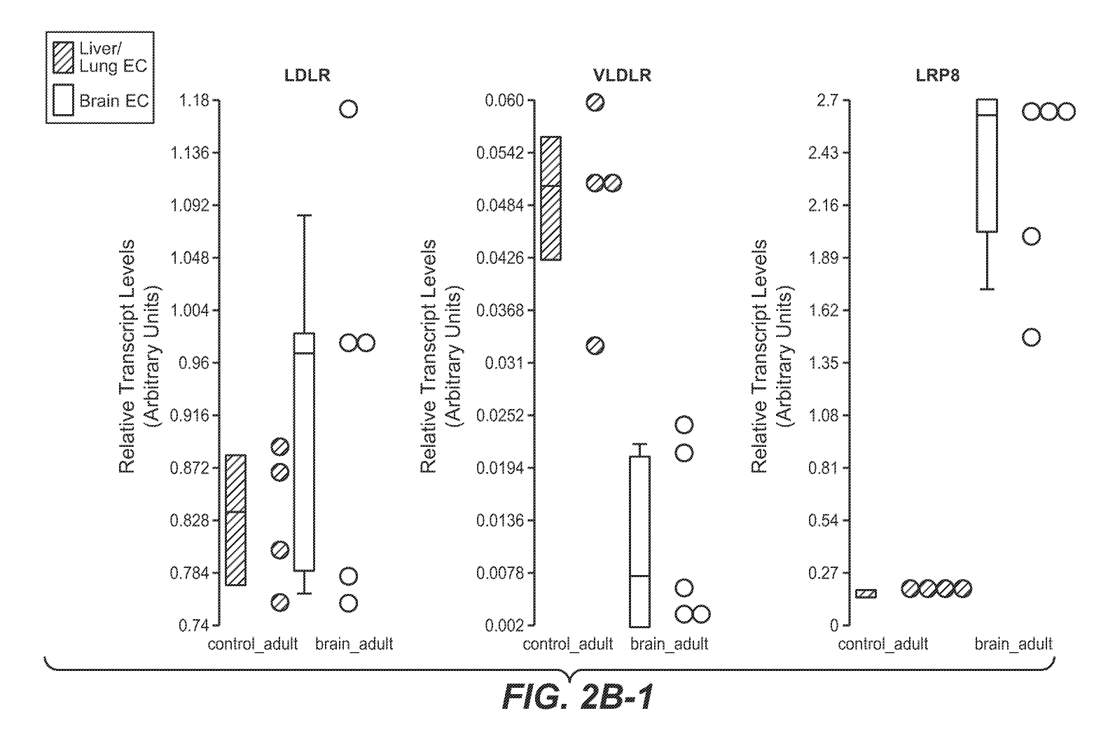

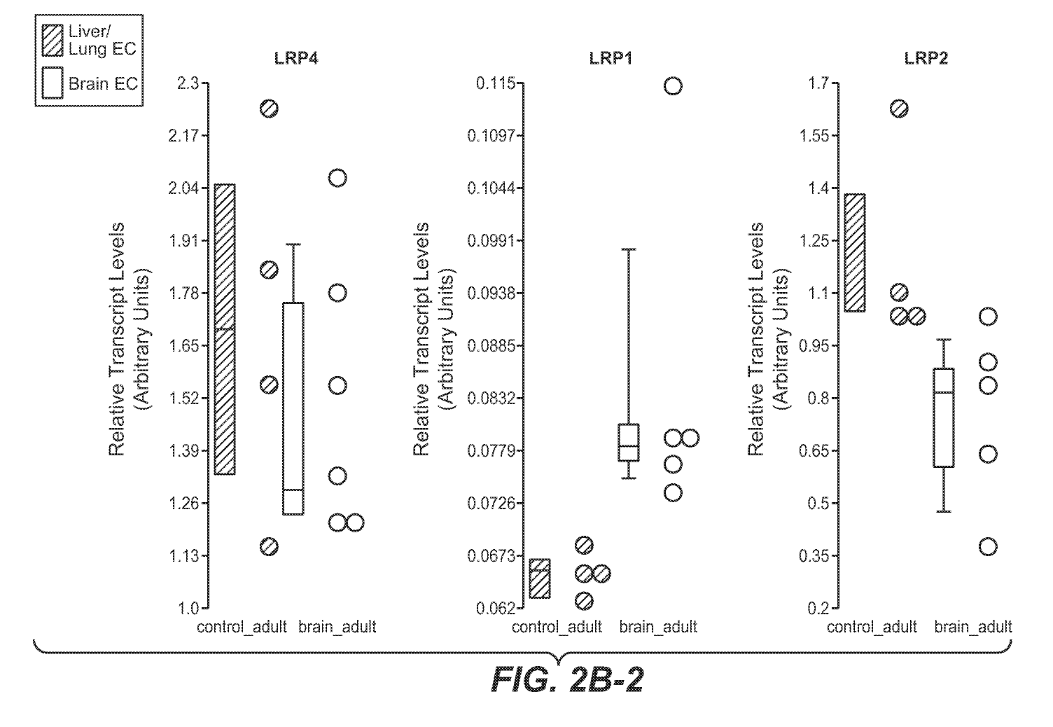

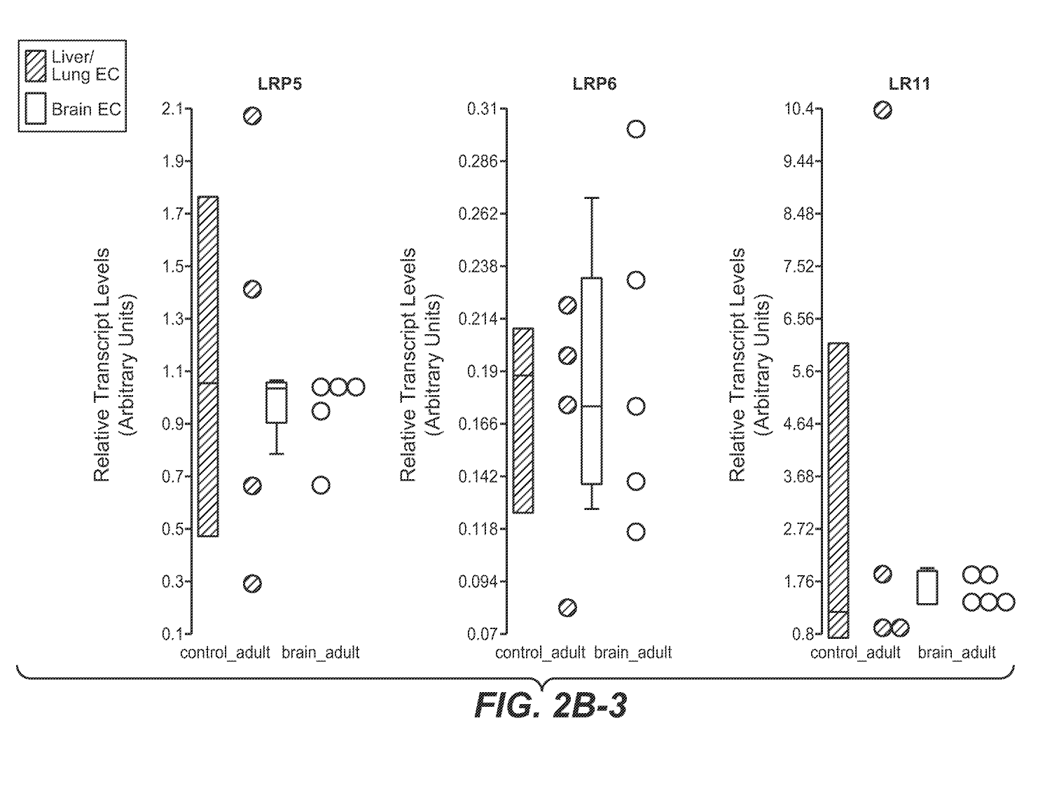

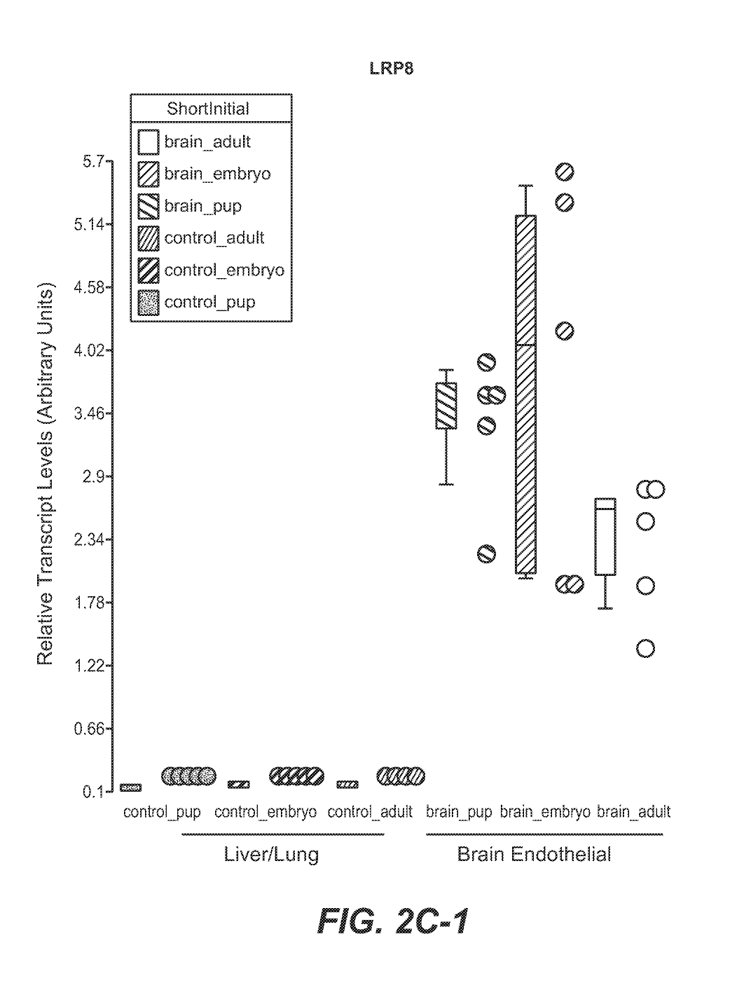

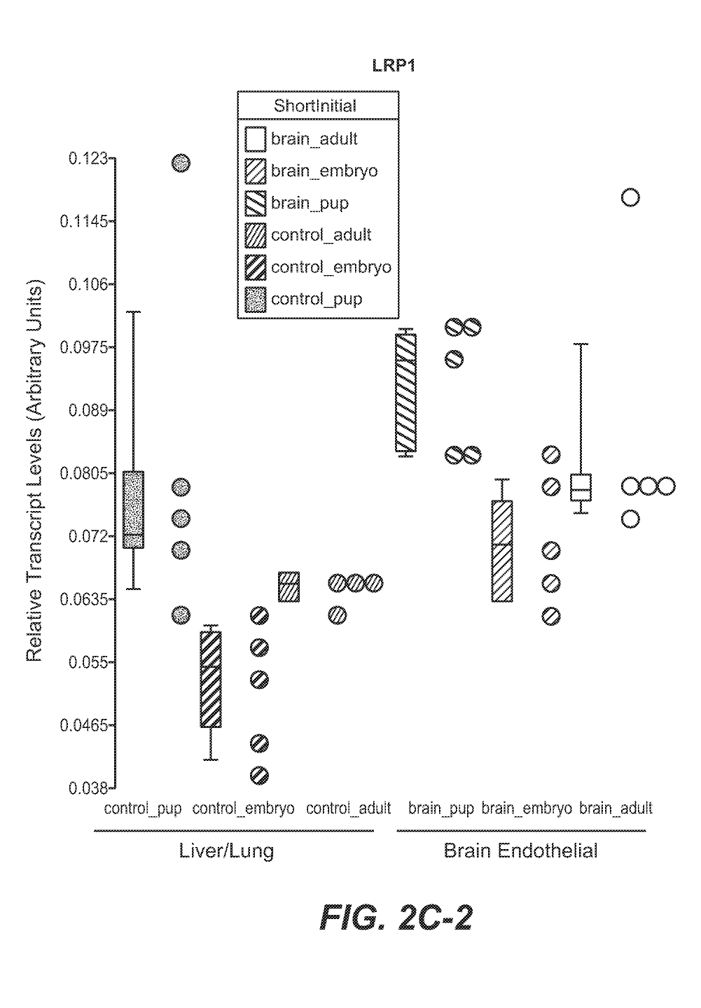

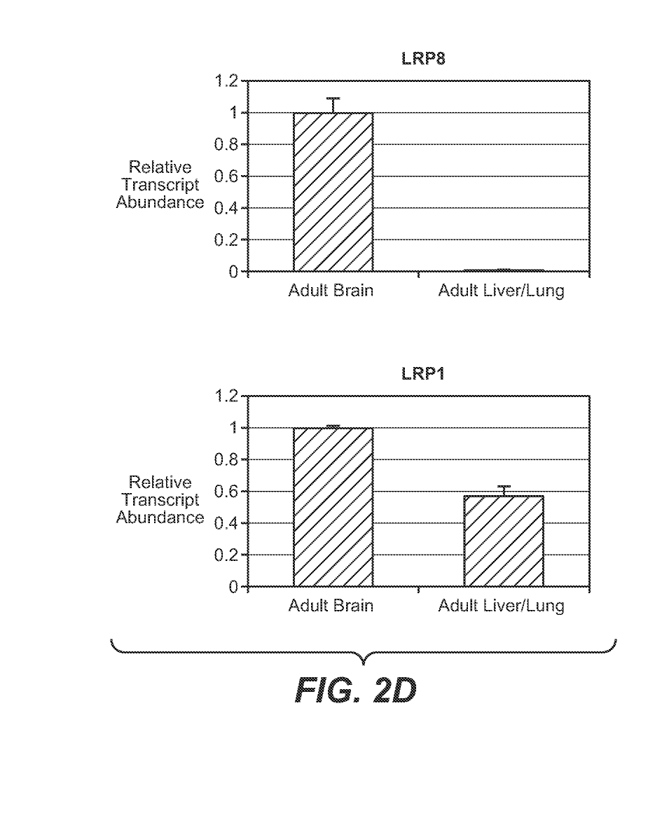

FACS sorting of endothelial cells for all cell suspensions was performed as follows. Cell suspensions were washed once with FACS buffer (2% BSA in PBS). 1:100 Fc preblock (CD16 or CD32) was added for 10 minutes at 4° C., followed by addition 1:100 of each of the two fluorescently labeled primary antibodies specific for cell markers (anti-mouse CD31 conjugated with FITC as an endothelial cell marker and anti-mouse CD45 conjugated with phycoerythrin as a marker of nucleated hematopoietic cells). The cells were washed twice with FACS buffer and filtered through a 70 micron filter centrifuged at 1,200 rpm for three minutes. Propidium iodide was added to a final concentration of 1 μg/ml to permit filtering of dead cells during FACS sorting. Approximately 0.8% of the brain cell samples were CD31-positive and CD45-negative endothelial cells, which RNA was purified by RNeasy Micro Plus kit (Qiagen) and were taken for further use in the microarray analyses (see FIG. 2A).

Quantitative RT-PCR was also performed using standard techniques for certain cell-specific markers to verify the above results. RT-PCR reactions were performed generally using the QuantiTect SYBR Green RT-PCR Kit One-step kit (Qiagen), with different cell specific primers as follows. To assess endothelial cell content, the marker Tie2 was measured using the Mm_Tek_{—}1 SG QuantiTect Primer Assay (Qiagen). To assess astrocyte cell content, the marker Aquaporin 4 was measured using the Mm_Aqp4_{—}1_SG QuantiTect Primer Assay (Qiagen). To assess oligodendrocyte content, the marker Sox10 was measured using the Mm_Sox10_{—}2_SG QuantiTect Primer Assay (Qiagen). To assess neuron content, the marker Snap25 was measured using the Mm_Snap25_{—}2_SG QuantiTect Primer Assay (Qiagen). To assess microglia content, the marker CD68 was measu [symbol] QuantiTect Primer Assay (Qiagen). All reactions were used with approximately 10 ng of template RNA (or 2 ng in the case of limited available starting material) and according to the manufacturer's directions. RT-PCR reactions were performed in 96-well plates in a Stratagene MX3000P PCR system.

For RNA microarray analyses, RNA concentrations were determined using a spectrophotometer (NanoDrop ND-1000). Total RNA was used to synthesize dsDNA and subjected to in vitro transcription in the presence of fluorescent dyes to produce labeled cRNA, using standard procedures. Cy5-labeled test sample RNAs and Cy3-labeled universal mouse reference RNAs were simultaneously hybridized onto commercial Agilent whole mouse gene expression arrays (WMG 4x44K) using an automated instrument (Tecan). Following hybridization, the arrays were washed, scanned and images were processed with feature extraction software (Agilent). The resulting data was analyzed using bioanalysis software (Partek Genomic Suite) and other automated microarray analyses. Quantitative RT-PCR was employed to confirm the above top two results. RT-PCR was performed as described above, but with the following primers (all from Qiagen): LRP8: QT00156100 and for comparison purposes LRP1: QT00155981.

The results are shown in FIGS. 2B-2D. While numerous low-density lipoprotein receptor family members were highly expressed in purified adult mouse blood-brain barrier endothelial cells, only LRP8 showed high expression in the brain endothelial cells and minimal expression in the lung/liver cells (FIGS. 2B-1 to 2B-3). Further, this high expression was found consistently in embryo, pup, and adult samples (FIGS. 2C-1 and 2C-2), while the expression in the lung/liver was also consistently minimal across all timepoints. These microarray results were confirmed by qRT-PCR using primers specific for the two best of these microarray hits, LRP8 and for comparison purposes LRP1 (FIG. 2D). While LRP1 was more highly expressed in brain endothelial cells than in lung/liver cells, the significant expression of LRP 1 in lung/liver cells while there was virtually no expression of LRP8 in lung/liver cells suggested that LRP8 was the preferred choice among the identified molecules for pursuing BBB-specific targeting and transport into the CNS across the BBB.

### Example 2

### Localization of LRP8 Expression in Endothelial Cells

The previous example demonstrated that LRP8 is highly and specifically expressed on endothelial cells at the BBB and that phage expressing LRP8 can translocate across a D3 cell monolayer in vitro. To better understand the role of LRP8 and whether it may be exploited for therapeutic delivery, experiments were performed to d [symbol] and tissue samples.

A. Identification and Characterization of Available Anti-LRP8 Antibodies

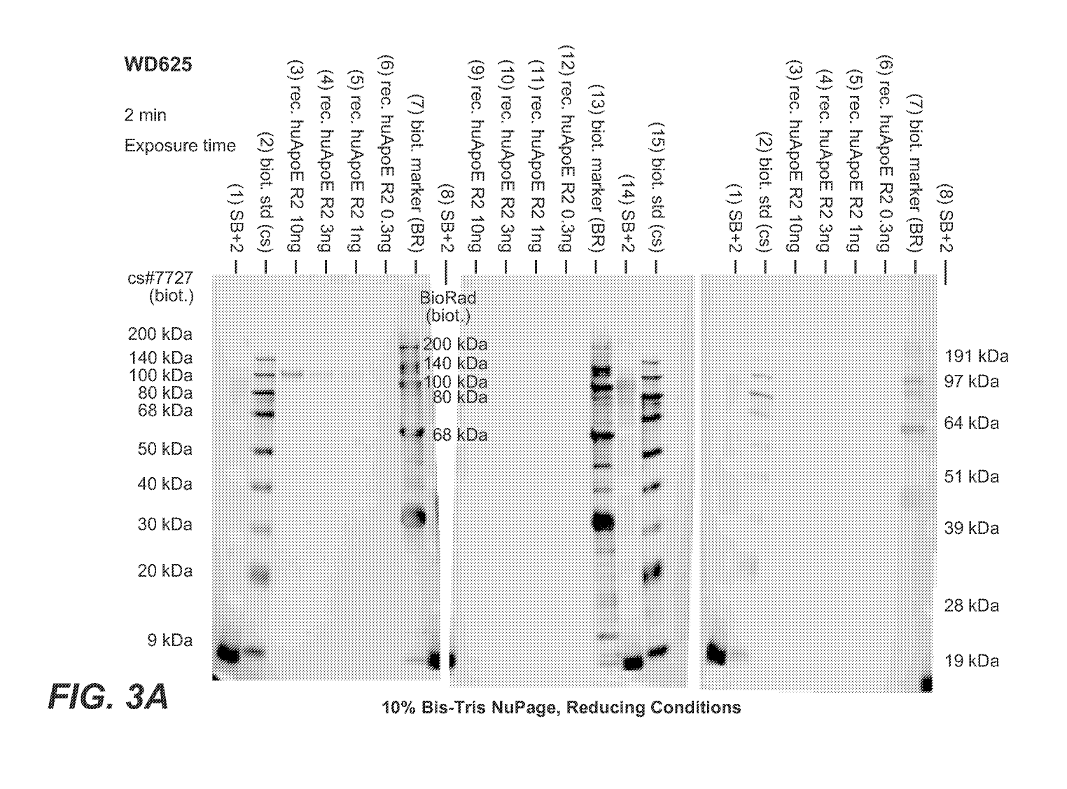

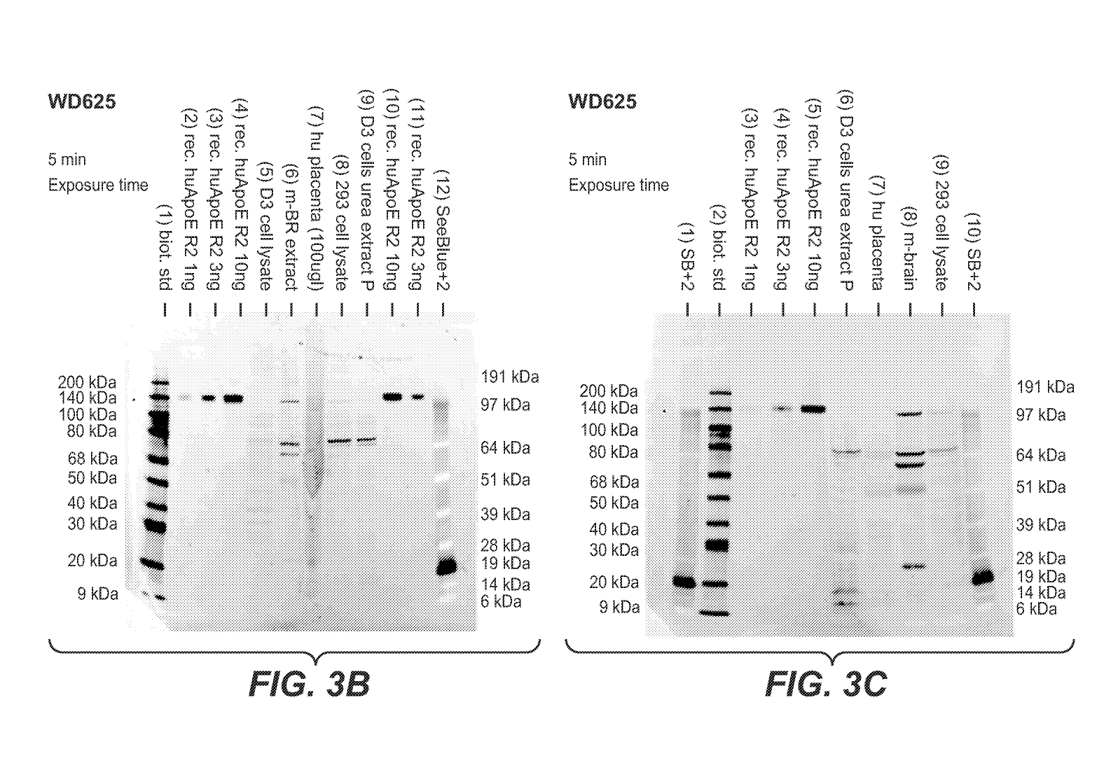

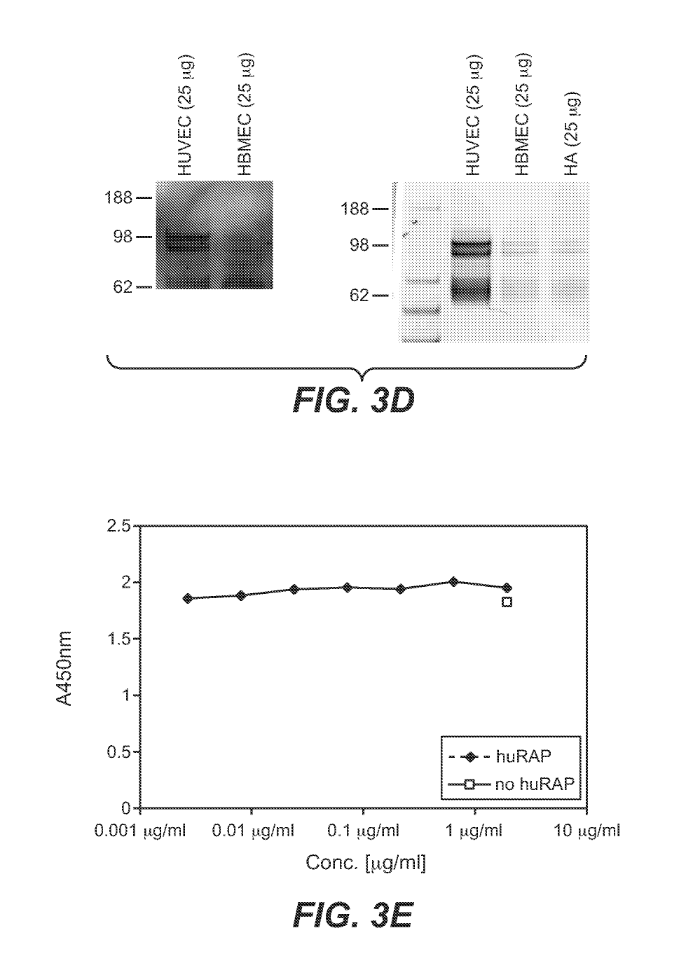

A survey of the binding of different commercially available monoclonal and polyclonal anti-LRP8 antibodies was performed by Western analysis using standard techniques to identify one or more antibodies able to recognize LRP8 in a purified state or in a complex mixture of proteins (i.e. a cell lysate). Recombinant human ApoER2 (R&D Systems #3520-AR) was diluted into Invitrogen LDS sample buffer with 1× reducing agent and loaded at 10 ng, 3 ng, 1 ng, and 0.3 ng into a 10% Bis-Tris Nupage 15-well gel (Invitrogen NP0303BOX). The gel was run in MOPS buffer for 50 min at room temperature, then transferred to the iBlot™ System (Millipore) and blocked for 2 hours with blocking buffer (Invitrogen). One blot (FIG. 3A, left panel) was incubated with mouse anti-ApoER2 monoclonal antibody (Abcam ab58216) as primary antibody at a dilution of 1:500 (2.0 μg/ml) for 48 hours at 4° C. on a rotator. After washing, the blot was incubated with biotinXX-anti-mouse secondary antibody at a dilution of 1:2000 for 2 hours at room temperature. Another blot (FIG. 3A, center panel) was incubated with rabbit anti-ApoER2 ployclonal primary antibody (Invitrogen 40-7800) for 48 hours at 4° C. on a rotator. After washing, the blot was incubated with biotinXX-anti-rabbit secondary antibody diluted 1:2000 for 2 hours at room temperature. A third blot (FIG. 3A, right panel) was incubated with mouse anti-LRP8 polyclonal primary antibody (Abnova H00007804-A01) at a dilution of 1:500 (2.0 μg/ml) for 48 hours at 4° C. on a rotator. After washing, the blot was incubated with biotinXX-anti-mouse secondary antibody diluted 1:2000 for 2 hours at room temperature. All blots were developed with Invitrogen QDot 625 streptavidin conjugate. FIG. 3A shows that the most sensitive detection of LRP8 of the three antibodies tested was observed in the leftmost blot.

To assess whether the Abcam anti-LRP8 antibody was able to detect LRP8 in cell lysates, further Western blot experiments were performed. D3 cells were grown in T175 flasks until passage 30, when the cells were scraped off, washed with PBS and lysed by the addition of 200 μl lysis buffer (20mM sodium phosphate, 500 mM NaCl, 2 mM MgCl₂, 4 mM OG, 1× complete) by soaking through a pipet tip. Samples were centrifuged at 70,000 rpm for 10 min, and the cleared lysate was added to 100 μl 7 M urea 0.1 M NaH₂PO₄ 100 mM Tris-HCl, 2 mM MgCl₂. To this mixture was added 0.5 μl 1 M MgCl₂ and 0.5 μl benzonase nuclease, followed by vortexing, and centrifugation 70,000 rpm for 10 min at 20° C. The resulting pellet was dissolved in urea and the extract and lysates were diluted into Invitrogen LDS sample buffer with 1× reducing agent. Samples were loaded onto a 4-12% NuPage Bis-Tris gel (Invitrogen) in various amounts, including: 10 ng, 3 ng, 1 ng, and 0.3 [symbol] lysate (mBR/293; FIG. 3B) or 36 μg lysate (hu-placenta/mBR/293; FIG. 3C). Gels were run in MOPS buffer at 200V for 50 min at room temperature, transferred to nitrocellulose in the iBlot System (Millipore) and blocked for 1 hour with blocking buffer (Invitrogen). The blots were incubated overnight with primary antibody Abcam anti-ApoER2 (#58216) diluted 1:500, 1 μg/ml at 4° C., followed by incubation with secondary antibody BiotinXX-anti-mouse diluted 1:2000 for 4 hours at room temperature. Blots were developed with Invitrogen QDot 625 streptavidin conjugate. The data clearly shows the presence of LRP8 in the D3 cell line (see FIGS. 3B and 3C).

The ability of certain of the antibodies to detect LRP8 in cell lysates was determined using similar assays in which samples of human umbilical vein endothelial cells (HUVEC) and human brain microvascular endothelial cells (HBMEC) were lysed in Triton lysis buffer in the presence of protease inhibitors (complete mini EDTA-free protease inhibitor tablets, Roche) for 30 minutes at 4° C. Incubation with the primary anti-ApoER2 antibodies (2 μg/ml Zymed 40-7800; 0.3 μg/ml Novus NB100-41391) was permitted to occur overnight at 4° C., and the secondary antibody (anti-goat or anti-rabbit antibody conjugated to horseradish peroxidase) was incubated for one hour at room temperature. Several commercially available antibodies were identified that were able to detect LRP8 in both cell extracts with varying sensitivity (see FIGS. 3B-3D), a few of which specifically recognized both human and mouse LRP8 (see, e.g., FIGS. 3B and 3C).

One antibody identified with these properties (mouse monoclonal anti-human ApoER2, Abcam #58216) was labeled with the fluorophore Cy5 using a Cy5 labeling kit (Amersham) according to the manufacturer's instructions. A standard ELISA assay was performed using plates with wells coated with 1 μg/ml recombinant human ApoER2 and various concentrations of anti-ApoER2, permitted to bind at 4° C. overnight. The plates were washed and treated with a secondary anti-mouse IgG antibody conjugated with horseradish peroxidase at a 1:10,000 dilution, and the plates were read. The results indicated that the IC50 for this particular anti-ApoER2 antibody was 68 ng/ml. To determine whether the antibody competes for binding to ApoER2 with RAP, another ELISA was performed as above, but instead of varying the concentration of anti-ApoER2, a consistent 1 μg/ml anti-ApoER2 was used in the presence or absence of varying amounts of recombinant human RAP. Specifically, Nunc-Immuno™ polystyrene Maxisorp 96-well plates (NUNC) were coated with 100 μl /well of 1 μg/ml recombinant human Apo ER2 (R&D Systems #3520-AR) in PBS overnight at 4° C. Two hundred microliters of 5% powdered milk (Bio Rad 170-0604) in T-PBS (0.1% Tween-20 Sigma 93773-250G) was added to each well for 2 hou [symbol] with wash buffer, human RAP (Innovative Research #IRAP) was added (50 μl/well, 0.003-2.0 μg/ml in 1% MP T-PBS) and preincubated for 30 min at room temperature. Anti-ApoER2 (abcam #58216) was added (50 μl/well to a final concentration of 1 μg/ml in 1% MP T-PBS) and the plates were incubated overnight at 4° C. Wells were washed as before and HRP-conjugated anti-mouse IgG (Sigma #A-9044) in 1% MP T-PBS (100 μl/well) was added to each well as per the manufacturer's suggested dilution, and wells were developed with a TMB substrate kit (Pierce #34021). The optical density at 450nm was measured using a Versamax Reader. As shown in FIG. 3E, no competition was observed between this antibody and rhRAP in this assay.

B. Analysis of LRP8 Distribution Within Endothelial Cells and in Various Tissues

Immunocytochemical and immunohistochemical analyses were performed to assess the distribution of LRP8 on human vascular endothelial cells and tissues from different sources.

1. HUVECs

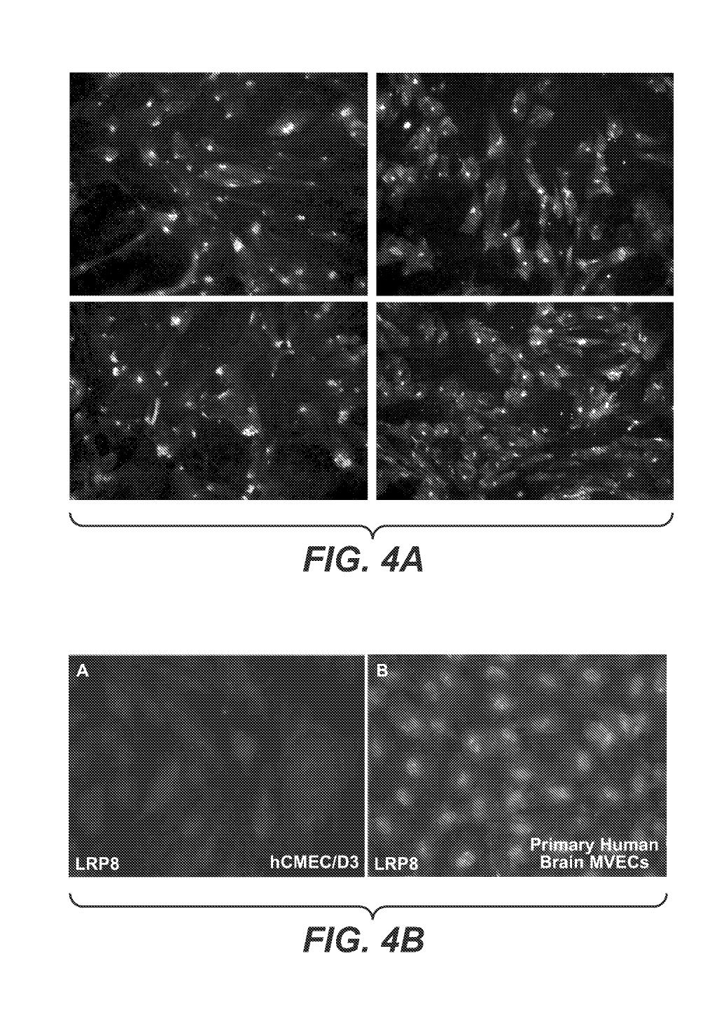

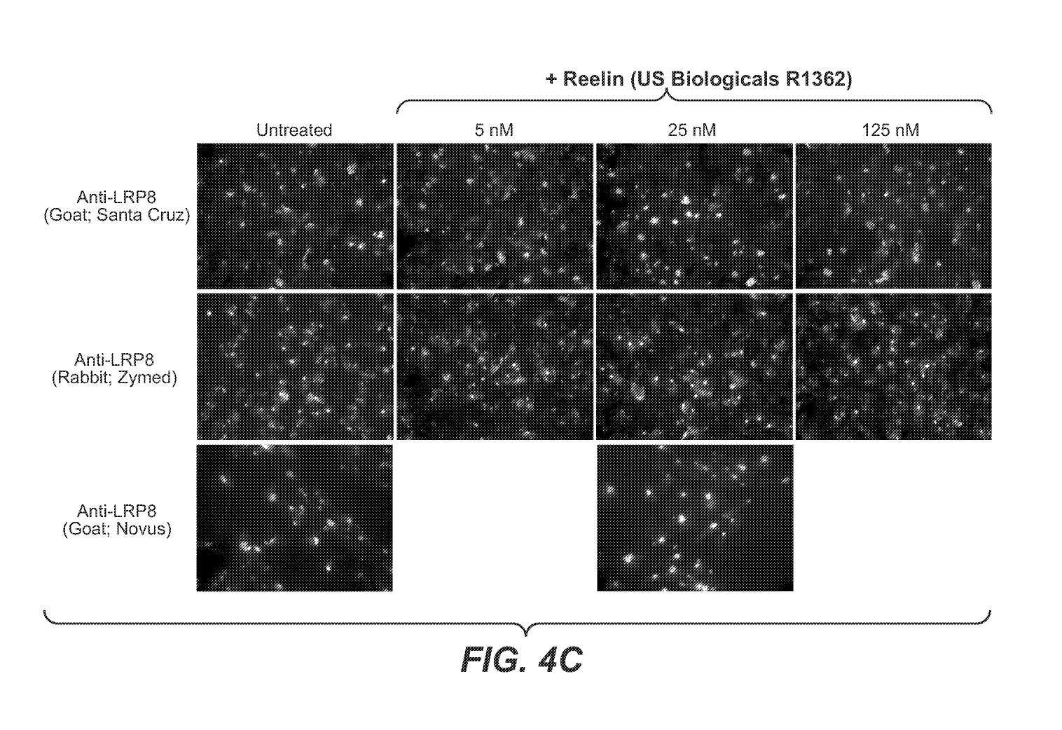

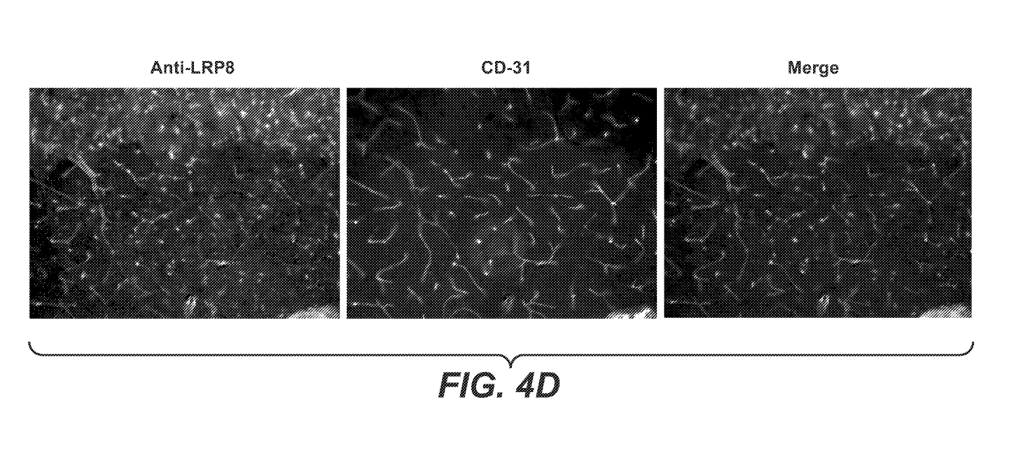

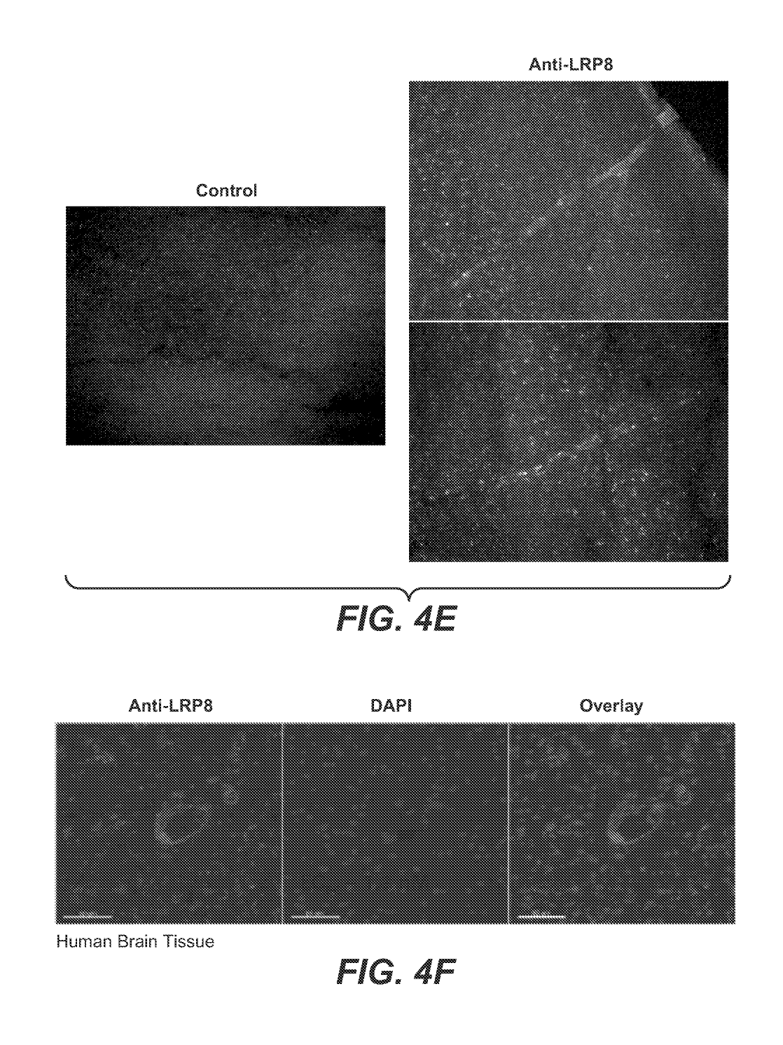

Immunocytochemical analyses were performed to assess the distribution of LRP8 on human vascular endothelial cells. Briefly, HUVEC cells were grown to a density of approximately 5,000 cells/cm². Cells were fixed in 4% PFA, washed twice with PBS, then blocked in 5% BSA +0.3% Triton-X100 in PBS for 30 minutes at room temperature. Primary anti-LRP8 antibody (Zymed 40-7800; Santa Cruz 10112) was added 1:100 to the cells in 1% BSA +0.3% Triton-X100 and incubated for two hours at room temperature. The cells were washed twice with PBS and an appropriate secondary anti-goat IgG or anti-rabbit IgG antibody (Alexa) was added 1:1000 in 1% BSA +0.3% Triton-X100. Immunofluorescence was observed with Axioplan 2 imaging (Zeiss). As shown in FIG. 4A, LRP8 staining in permeabilized HUVEC appeared punctuate, but consistent staining in all cells was observed. These experiments were repeated but with HUVECs that had been subjected to a 30 minute treatment with 5 nM, 25 nM or 125 nM of the LRP8 ligand reelin (US Biologicals) at 37° C. prior to washing in PBS and subsequent fixation and immunolabeling as described above. Untreated HUVEC displayed the same staining pattern as HUVEC that had been pretreated with any of the concentrations of reelin, indicating that reelin did not have an effect on LRP8 localization in HUVEC (FIG. 4C).

2. hCMEC/D3 and Primary Human Brain Endothelial Cells

Monolayers of primary human brain endothelial cells (CellSystems) and hCMEC/D3 cells grown to confluence on collagen-coated cover slips were fixed in methanol at −20° C. for 10 min. Cells were incubated for 1 hour in 1× PBS containing 1.5% BSA at room temperature, permeabilised for 10 min (1× PBS with 0.1% Triton X [symbol] human ApoER2 antibody from Sigma (A3481) or Abcam (ab52905) for 1 hour at room temperature. Cells were subjected to 1× PBS washes for 15 min and then incubated with goat anti-rabbit IgG-Alexa Flour 488 (1:200, Invitrogen) for 1 hour at room temperature. Cells were washed in 1× PBS for 30 min and cover slips mounted in U1traCruz™ fluorescent mounting medium (Santa Cruz) and analysed by fluorescent microscopy. The results are shown in FIG. 4B. Both images show a membrane and vesicular distribution of LRP8; the expression level is higher in primary cells (right panel) than in the immortalized cell line (left panel).

3. Mouse and Human Brain Tissue

Similar studies were performed on brain tissue in mice. First, LRP8 visualization in tissue in vitro was assessed. The experiments were performed as follows. Brains from C57b16 mice were harvested, perfused with PBS, and fixed in 4% PFA for 48 hours at 4 ° C. Tissues were then incubated in a 30% sucrose solution for 24 hours at 4 ° C. Primary rabbit anti-human LRP8 antibody (Zymed 40-7800) and anti-CD31 antibody (BD Biosciences) were each added at a 1:500 dilution and incubated overnight at 4 ° C. The tissues were washed with multiple changes of PBS, a secondary anti-rabbit or anti-goat IgG antibody (Alexa) was added at a dilution of 1:200, and samples were incubated for 2 hours at room temperature. Immunofluorescence images were visualized and assessed with an Axioplan 2 Imaging microscope and software (Zeiss). As shown in FIG. 4D, LRP8 staining colocalized with CD31 staining. Since CD31 is a known vascular endothelial cell marker, this suggests that LRP8 is expressed on such cells in vivo, and that LRP8 staining is specific for CD31-positive vascular endothelial cells, such as would be found at the blood-brain barrier.

To determine whether systemically administered anti-LRP8 antibody could be detected in the brain, mice were injected intravenously with labeled antibody and the presence of the antibody in the brain was assessed. Briefly, mice were intravenously injected via the tail vein with Alexa488-conjugated anti-LRP8 antibody (Abcam) or an Alexa488-conjugated control immunoglobulin at a dose of 2.5 mg/kg. After one hour, the mice were perfused with PBS, and the brains were extracted and immediately frozen without fixation. The brains were sectioned (40 micron sections) on a cryostat, and the sections were mounted on slides with fluorogold. Immunofluorescence images were visualized and assessed with an Axioplan 2 Imaging microscope and software (Zeiss). As shown in FIG. 4E, little to no staining was observed in the control antibody image (left panel), while significant staining was observed in the anti-LRP8 antibody images (right panels). Blood vessels were heavily stained (as expected due to the presence of LRP8 at the blood brain barrier), but significant staining was also observed throughout the brain tissue in a punctate pattern. This [symbol] administered by tail vein injection circulated and was transported across the BBB into the mouse brain.

The immunohistochemistry data shown in FIG. 4F was performed using cryostat sections of unfixed human brain tissue from a cortical brain region obtained postmortem which was labeled by indirect immunofluorescence. A successive two-step incubation was used to detect rabbit polyclonal anti-LRP8 antibody (Sigma), involving affinity-purified goat anti-rabbit IgG (H+L) conjugated to Alexa555 (Molecular Probes). Sectioning, staining and fluorescence microscopy was done according to standard procedures together with DAPI staining to identify cell nuclei. LRP8 staining coincident with blood vessels was readily detected.

### Example 3

### In vitro Transmigration Assay

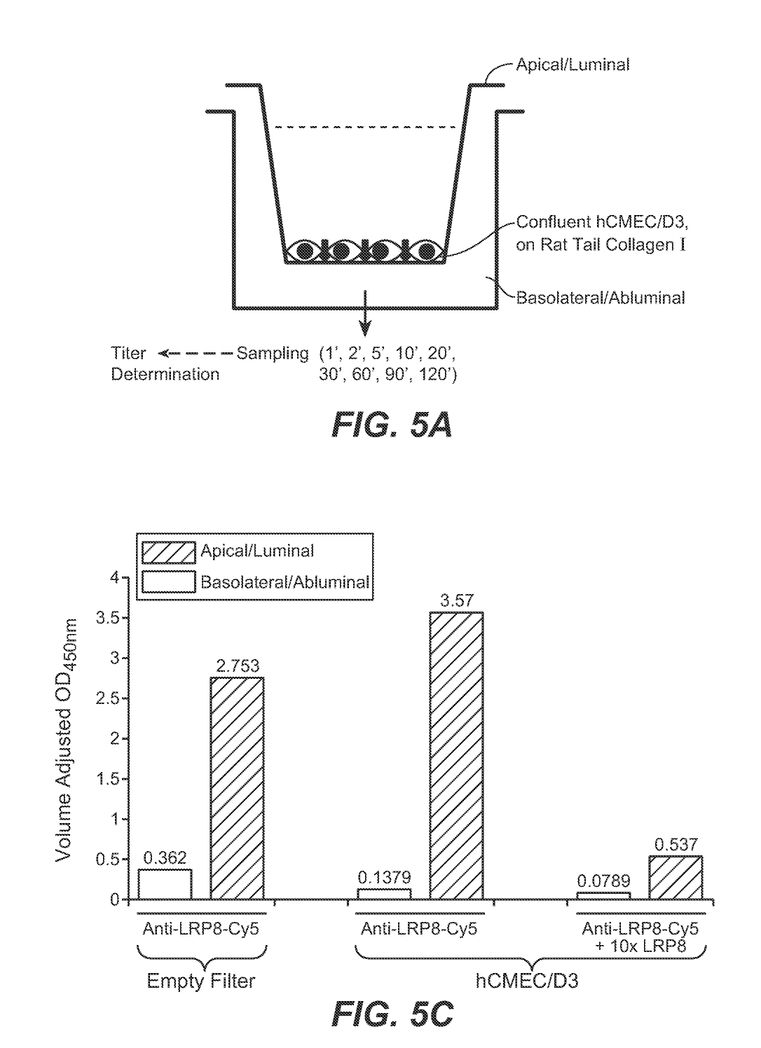

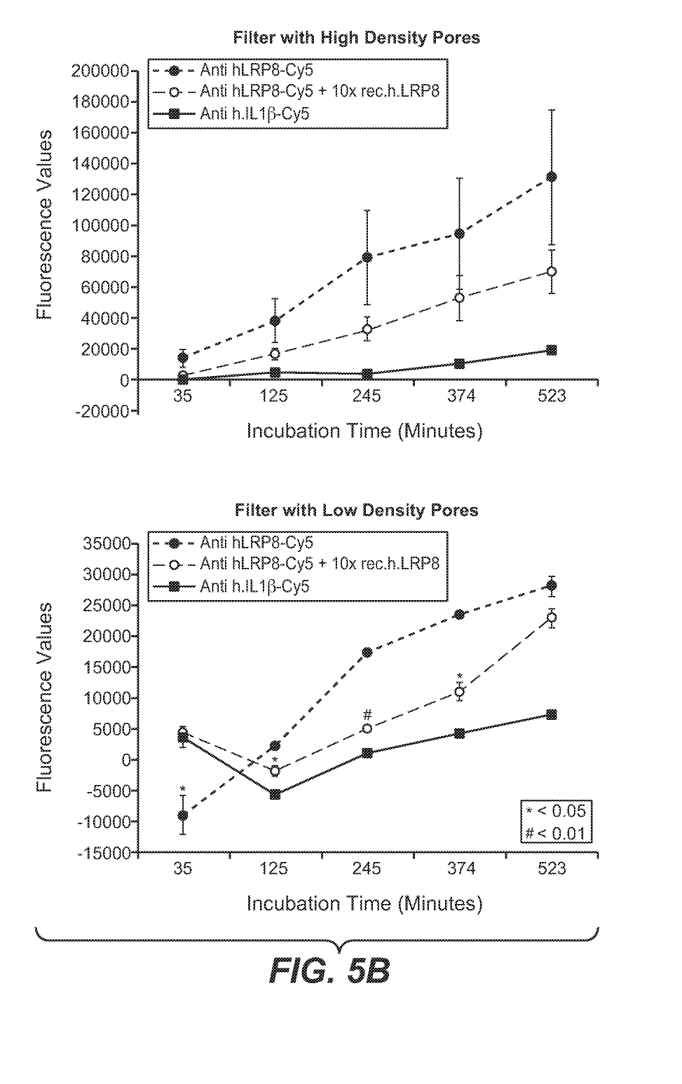

The previous examples demonstrate that LRP8 is highly and specifically expressed on vascular endothelial cells at the blood brain barrier both in vitro and in vivo. To assess the ability of LRP8 to translocate an associated protein across a vascular endothelial cell layer, transmigration assays were performed. The experimental setup is described schematically in FIG. 5A. Briefly, hCMEC/D3 cells were plated in transwell plates using a Corning 0.4 μm transwell (#3460) with a growth area of 1.1 cm² on a 12-well culture plate precoated with 10 μg/ml collagen (rat tail collagen type 1 #35436) in 0.02N acetic acid. Cells were plated at a density of 2.5×10⁴ cells/ml in resting media (EGM2 basal medium; Lonza CC-4176) containing 12.5% human serum and 2.2 μM hydrocortisone (Sigma #H0888) in a total volume of 0.5 ml/well (1.5 ml basolateral). The medium was changed after days 3 and 6. The cells were confluent at day 8. Cy5-labeled anti-LRP8 antibody was prepared using a Cy5 mAb Labeling Kit (Amersham #PA35001) and added to a concentration of 0.23 mg/ml in 500 μl medium on the apical side of the transwell plate. A Cy5-labeled control antibody (anti-IL1β) was prepared identically using the same kit and also added to the apical side.

For the transmigration experiments, anti-LRP8-Cy5 antibody (final concentration of 0.01 μM, 1.5 μg/ml) or anti-LRP8-Cy5 antibody premixed with a 10-fold excess of recombinant human ApoER2 (final concentration 0.11 μM, 10 μg/ml) were added to the D3 cells on the apical side of transwell. Two types of transwell setups were assessed: filters with high density pores and filters with low density pores. Anti-IL1β-Cy5 antibody controls were performed using identical concentrations. Filter-only controls (collagen-coated) lacking D3 cells were used to measure background transport. Aliquots (100 μl) of the medium from the basolateral side of the transwell plate were removed at [symbol] anti-LRP8 and anti-IL1β content using the EnVision™ Fluorescence Reader (DakoCytomation) using an excitation wavelength of 620 nm and an emission wavelength of 685 nm in a 96-well black plate prior to replacement of the samples into the basolateral chamber.

The results in FIG. 5B show that anti-LRP8 antibody transmigrated across the D3 cell layer, and that this transmigration was reduced in the presence of excess soluble LRP8 competitor. Very little transport of the anti-IL1β antibody across the D3 cell layer was observed. These results clearly indicate the need for specific binding to LRP8 in this system to promote transport.

FIG. 5C shows an ELISA experiment performed similarly to the ELISAs in Example 2. Plates were coated with 1 μg/m1 recombinant human ApoER2 (R&D Systems #3520-AR) diluted into PBS to a volume of 100 μl/well and incubated overnight at 4° C. Wells were blocked by the addition of 200 μl 5% milk powder (BioRad 170-6404) diluted into T-PBS (0.1% Tween-20, Sigma #93773-250G) and plates were incubated for 2 hours at room temperature. The wells were washed and 100 μl/well of basolateral medium from the experiment above containing anti-LRP8-Cy5 with or without the addition of LRP8 was added, followed by an overnight incubation at 4° C. The wells were washed, and 100 μl/well HRP-conjugated anti-mouse IgG (Sigma #A-9044) diluted into 1% MP T-PBS was added. Plates were developed using the TMB substrate kit (Pierce #34021) according to the manufacturer's instructions and measured in the VersaMax™ reader (Molecular Devices) at 450 nm. The results (FIG. 5C) are in agreement with the data in FIG. 5B which shows that anti-LRP8 antibody is transported across the D3 cell layer, and further that a greater amount of the anti-LRP8 antibody is transported across the D3 cell layer if there is no competition with soluble LRP8. The observed low value for the apical sample with 10-fold excess of LRP8 is due to interference in the ELISA assay.

### Example 4

### In vivo Transmigration Assay

The previous examples demonstrate that an anti-LRP8 antibody binds to LRP8 and is translocated across the BBB in vitro. Experiments are performed to assess the tissue distribution of labeled anti-LRP8 antibody following a single IV dose in vivo in a mouse model. Briefly, C57B1/6 mice are injected with ¹²⁵I-labeled anti-LRP8 in the presence or absence of a ¹³¹I-labeled control antibody that does not bind to LRP8. Two mice of each group are sacrificed at post-injection timepoints of 30 minutes, 1 hour, 4 hours, 12 hours and 24 hours, and blood and tissues are collected for analysis [symbol] label.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A composition comprising an LRP8-binding molecule and at least one CNS-active compound.

2. The composition of claim 1, wherein the LRP8-binding molecule is conjugated to the at least one CNS-active compound.

3. The composition of claim 2, wherein the conjugation is a covalent linkage between the LRP8-binding molecule and the at least one CNS-active compound.

4. The composition of claim 2, wherein the conjugation is by a linker.

5. The composition of claim 1, wherein the LRP8-binding molecule is selected from a natural ligand of LRP8, a fragment of a natural ligand of LRP8, a modified form of a natural ligand of LRP8, and a fragment of a modified form of a natural ligand of LRP8.

6. The composition of claim 5, wherein the natural ligand of LRP8 is selected from reelin and selenoprotein P.

7. The composition of claim 1, wherein the LRP8-binding molecule is an antibody.

8. The composition of claim 7, wherein the antibody is a multispecific antibody.

9. The composition of claim 7, wherein the antibody is selected from a monoclonal antibody, a human antibody, a humanized antibody, a chimeric antibody, and an antibody fragment that binds LRP8.

10. The composition of claim 1, wherein the LRP8-binding molecule does not compete with one or more natural ligands of LRP8 for binding to LRP8.

11. The composition of claim 1, wherein the LRP8-binding molecule competes with one or more natural ligands of LRP8 for binding to LRP8.

12. The composition of claim 1, wherein the LRP8-binding molecule binds to the extracellular domain of LRP8.

13. The composition of claim 1, wherein the LRP8-binding molecule preferentially binds to LRP8 expressed in the brain.

14. The composition of claim 1, wherein the CNS-active compound is selected from a therapeutic compound and a diagnostic compound.

15. The composition of claim 14, wherein the therapeutic compound is selected from a neurotrophic factor and a compound to treat or prevent one or more of neuropathy, amyloidosis, cancer, an ocular disease or disorder, viral or microbial infection, inflammation, ischemia, neurodegenerative disease, seizure, a behavioral disorder, and a lysosomal storage disease.

16. The composition of claim 15, wherein the therapeutic compound is selected from a compound to treat Parkinson's disease and a compound to treat Alzheimer's disease.

17. The composition of claim 15, wherein the diagnostic compound is a labeled peptide or antibody that specifically binds to a CNS target.

18. The composition of claim 1, wherein binding of the LRP-binding molecule to LRP8 effects the transport of the CNS-active compound across the blood-brain barrier.

19. A pharmaceutical formulation comprising the composition of claim 1 and a pharmaceutically acceptable carrier.

20. The pharmaceutical formulation of claim 19, further comprising an additional therapeutic agent.

21-32. (canceled)

33. A method for modulating the transport of a CNS-active compound across the blood-brain barrier in a mammal by modulating the expression, stability, or activity of LRP8.

34. A method for modulating the transport of a CNS-active compound across a vascular endothelial cell layer including tight junctions by targeting LRP8.

35. The method of claim 33, wherein the targeting is by means of an LRP8-binding molecule and transport of the CNS-active compound is increased.

36. The method of claim 35, wherein the LRP8-binding molecule and the CNS-active compound are administered to the mammal simultaneously.

37. The method of claim 36, wherein the LRP8-binding molecule is conjugated to the CNS-active compound.

38. The method of claim 37, wherein the conjugation between the LRP8-binding molecule and the CNS-active compound is selected from covalent association, association with a linker, and as different binding moieties within the same multispecific antibody.

39. The method of claim 37, wherein the LRP8-binding molecule is selected from a natural ligand of LRP8, a fragment of a natural ligand of LRP8, a modified form of a natural ligand of LRP8, and a fragment of a modified form of a natural ligand of LRP8.

40. The method of claim 39, wherein the natural ligand of LRP8 is selected from reelin and selenoprotein P.

41. The method of claim 33, wherein the LRP8-binding molecule is an antibody.

42. The method of claim 41, wherein the antibody is a multispecific antibody.

43. The method of claim 41, wherein the antibody is selected from a monoclonal antibody, a human antibody, a humanized antibody, a chimeric antibody, and an antibody fragment that binds LRP8.

44. The method of claim 33, wherein the LRP8-binding molecule does not compete with one or more natural ligands of LRP8 for binding to LRP8.

45. The method of claim 33, wherein the LRP8-binding molecule competes with one or more natural ligands of LRP8 for binding to LRP8.

46. The method of claim 33, wherein the LRP8-binding molecule binds to the extracellular domain of LRP8.

47. The method of claim 33, wherein the LRP8-binding molecule preferentially binds to LRP8 expressed in the brain.

48. The method of claim 33, wherein the CNS-active compound is selected from a therapeutic compound and a diagnostic compound.

49. The method of claim 48, wherein the therapeutic compound is selected from a neurotrophic factor and a compound to treat or prevent one or more of neuropathy, amyloidosis, cancer, an ocular disease or disorder, viral or microbial infection, inflammation, ischemia, neurodegenerative disease, seizure, a behavioral disorder, and a lysosomal storage disease.

50. The method of claim 49, wherein the therapeutic compound is selected from a compound to treat Parkinson's disease and a compound to treat Alzheimer's disease.

51. The method of claim 49, wherein the diagnostic compound is a labeled peptide or antibody that specifically binds to a CNS target.

52. The method of claim 33, wherein the mammal is a human.

53. The method of claim 36, wherein the LRP8-binding molecule and the CNS-active compound are administered in conjunction with one or more additional therapeutic agents.

54. The method of claim 36, wherein the LRP8-binding molecule and the CNS-active compound are administered in conjunction with a pharmaceutically-acceptable carrier.

55. A method of treating an individual having a CNS disease or CNS disorder comprising administering to the individual an effective amount of the composition of claim 1.

56. A method of decreasing or preventing the severity, duration, or symptoms of a CNS disease or CNS disorder in an individual suffering therefrom comprising administering to the individual an effective amount of the composition of claim 1.

57. A method of diagnosing a CNS disease or CNS disorder in an individual comprising administering to the individual an effective amount of the composition of claim 1, visualizing or quantifying the CNS-active compound in the brain of the individual, and comparing the results to control results from individuals with known instance of the CNS disease or disorder or lack thereof.

58. A method of staging a CNS disease or CNS disorder in an individual comprising administering to the individual an effective amount of the composition of claim 1, visualizing or quantifying the CNS-active compound in the brain of the individual, and comparing the results to control results from individuals with known stages of the CNS disease or CNS disorder.


SEQUENCE LISTING (4 sequences)

SEQ ID NO: 1 - protein, 963 aa, Homo sapiens
MGLPEPGPLRLLALLLLLLLLLLLRLQHLAAAAADPLLGGQGPAKECEKDQFQCRNERCIPSVWRCDEDDDCLDHSDEDDCPKKTCADSDFTCDNGHCIHERWKCDGEEECPDGSDESEATCTKQVCPAEKLSCGPTSHKCVPASWRCDGEKDCEGGADEAGCATLCAPHEFQCGNRSCLAAVFVCDGDDDCGDGSDERGCADPACGPREFRCGGDGGGACIPERWVCDRQFDCEDRSDEAAELCGRPGPGATSAPAACATVSQFACRSGECVHLGWRCDGDRDCKDKSDEADCPLGTCRGDEFQCGDGTCVLAIKHCNQEQDCPDGSDEAGCLQGLNECLHNNGGCSHICTDLKIGFECTCPAGFQLLDQKTCGDIDECKDPDACSQICVNYKGYFKCECYPGYEMDLLTKNCKAAGGKSPSLIFTNRYEVRRIDLVKRNYSRLIPMLKNVVALDVEVATNRIYWCDLSYRKIYSAYMDKASDPKEQEVLIDEQLHSPEGLAVDWVHKHIYWTDSGNKTISVATVDGGRRRTLFSRNLSEPRAIAVDPLRGFMYWSDWGDQAKIEKSGLNGVDRQTLVSDNIEWPNGITLDLLSQRLYWVDSKLHQLSSIDFSGGNRKTLISSTDFLSHPFGIAVFEDKVFWTDLENEAIFSANRLNGLEISILAENLNNPHDIVIFHELKQPRAPDACELSVQPNGGCEYLCLPAPQISSHSPKYTCACPDTMWLGPDMKRCYRAPQSTSTTTLASTMTRTVPATTRAPGTTVHRSTYQNHSTETPSLTAAVPSSVSVPRAPSISPSTLSPATSNHSQHYANEDSKMGSTVTAAVIGIIVPIVVIALLCMSGYLIWRNWKRKNTKSMNFDNPVYRKTTEEEDEDELHIGRTAQIGHVYPAAISSFDRPLWAEPCLGETREPEDPAPALKELFVLPGEPRSQLHQLPKNPLSELPVVKSKRVALSLEDDGLP

SEQ ID NO: 2 - protein, 931 aa, Homo sapiens
AADPLLGGQGPAKECEKDQFQCRNERCIPSVWRCDEDDDCLDHSDEDDCPKKTCADSDFTCDNGHCIHERWKCDGEEECPDGSDESEATCTKQVCPAEKLSCGPTSHKCVPASWRCDGEKDCEGGADEAGCATLCAPHEFQCGNRSCLAAVFVCDGDDDCGDGSDERGCADPACGPREFRCGGDGGGACIPERWVCDRQFDCEDRSDEAAELCGRPGPGATSAPAACATVSQFACRSGECVHLGWRCDGDRDCKDKSDEADCPLGTCRGDEFQCGDGTCVLAIKHCNQEQDCPDGSDEAGCLQGLNECLHNNGGCSHICTDLKIGFECTCPAGFQLLDQKTCGDIDECKDPDACSQICVNYKGYFKCECYPGYEMDLLTKNCKAAGGKSPSLIFTNRYEVRRIDLVKRNYSRLIPMLKNVVALDVEVATNRIYWCDLSYRKIYSAYMDKASDPKEQEVLIDEQLHSPEGLAVDWVHKHIYWTDSGNKTISVATVDGGRRRTLFSRNLSEPRAIAVDPLRGFMYWSDWGDQAKIEKSGLNGVDRQTLVSDNIEWPNGITLDLLSQRLYWVDSKLHQLSSIDFSGGNRKTLISSTDFLSHPFGIAVFEDKVFWTDLENEAIFSANRLNGLEISILAENLNNPHDIVIFHELKQPRAPDACELSVQPNGGCEYLCLPAPQISSHSPKYTCACPDTMWLGPDMKRCYRAPQSTSTTTLASTMTRTVPATTRAPGTTVHRSTYQNHSTETPSLTAAVPSSVSVPRAPSISPSTLSPATSNHSQHYANEDSKMGSTVTAAVIGIIVPIVVIALLCMSGYLIWRNWKRKNTKSMNFDNPVYRKTTEEEDEDELHIGRTAQIGHVYPAAISSFDRPLWAEPCLGETREPEDPAPALKELFVLPGEPRSQLHQLPKNPLSELPVVKSKRVALSLEDDGLP

SEQ ID NO: 3 - protein, 3460 aa, Homo sapiens [sequence omitted]

SEQ ID NO: 4 - protein, 381 aa, Homo sapiens
  Unsure 59,300,318,330,345,352,367,369,376,378: Unknown amino acid
MWRSLGLALALCLLPSGGTESQDQSSLCKQPPAWSIRDQDPMLNSNGSVTVVALLQASXYLCIIEASKLEDLRVKLKKEGYSNISYIVVNHQGISSRLKYTHLKNKVSEHIPVYQQEENQTDVWTLLNGSKDDFLIYDRCGRLVYHLGLPFSFLTFPYVEEAIKIAYCEKKCGNCSLTTLKDEDFCKRVSLATVDKTVETPSPHYHHEHHHNHGHQHLGSSELSENQQPGAPNAPTHPAPPGLHHHHKHKGQHRQGHPENRDMPASEDLQDLQKKLCRKRCINQLLCKLPTDSELAPRSXCCHCRHLIFEKTGSAITXQCKENLPSLCSXQGLRAEENITESCQXRLPPAAXQISQQLIPTEASASXRXKNQAKKXEXPSN